SEQUENCE LISTING (5 sequences)

SEQ ID NO: 1 - protein, 4 aa, synthetic construct
  MOD_RES 2: note = D-phenylalanine
HFRW

SEQ ID NO: 2 - protein, 4 aa, synthetic construct
HFRW

SEQ ID NO: 3 - protein, 7 aa, synthetic construct
  MOD_RES 1: note = Norleucine
  MOD_RES 4: note = D-2-napthylalanine
XDHXRWK

SEQ ID NO: 4 - protein, 7 aa, synthetic construct
  MOD_RES 1: note = Norleucine
  MOD_RES 4: note = D-2-napthylalanine
XDPXRWK

SEQ ID NO: 5 - protein, 13 aa, synthetic construct
  MOD_RES 4: note = Norleucine
  MOD_RES 6: note = Octahydroindole-2-carboxylic Acid
  MOD_RES 7: note = D- 4-phenyl-phenylalanine
  MOD_RES 8: note = Pipecolic acid
SYSXEXXXWGKPV

(19) United States
(12) Patent Application Publication
(10) Pub. No.: US 2024/0002438 A1
(43) Pub. Date: Jan. 4, 2024

(54) MELANOCORTIN LIGANDS AND METHODS OF USE THEREOF

(71) Applicants: REGENTS OF THE UNIVERSITY OF MINNESOTA, Minneapolis, MN (US); FLORIDA INTERNATIONAL UNIVERSITY, Miami, FL (US); NOVA SOUTHEASTERN UNIVERSITY, Fort Lauderdale, FL (US)

(72) Inventors: Carrie Haskell-Luevano, MINNEAPOLIS, MN (US); Mark David Ericson, MINNEAPOLIS, MN (US); Richard A. Houghten, MIAMI, FL (US); Clemencia Pinilla, MIAMI, FL (US); Marcello Angelo Giulianotti, MIAMI, FL (US); Radleigh G. Santos, FORT LAUDERDALE, FL (US)

(21) Appl. No.: 17/944,574

(22) Filed: Sep. 14, 2022

Related U.S. Application Data
(60) Provisional application No. 63/244,135, filed on Sep. 14, 2021.

(51) Int. Cl. C07K 5/117; C07K 5/107

(52) U.S. Cl. CPC C07K 5/1024; C07K 5/1016; A61K 38/00

(57) ABSTRACT

Certain embodiments of the invention provide a compound of formula (I) or a compound of formula (II):

R¹—A¹—A²—A³—A⁴—N(R²)₂ (I)

R³—A⁵—A⁶—A⁷—A⁸—N(R⁴)₂ (II)

or a salt thereof, wherein R¹, R², R³, R⁴, A¹, A², A³, A⁴, A⁵, A⁶, A⁷, and A⁸ are as defined herein, as well as methods of use thereof.

### CROSS-REFERENCE TO RELATED APPLICATIONS

The present application claims priority to U.S. Provisional Patent Application No. 63/244,135, filed Sep. 14, 2021, the entire contents of which are hereby incorporated by reference.

### GOVERNMENT FUNDING

This invention was made with government support under DK091906 and DK124504 awarded by the National Institutes of Health. The government has certain rights in the invention.

### BACKGROUND OF THE INVENTION

Melanocortin receptors have been linked to a number of biological processes. In particular, the melanocortin-4 receptor is involved in appetite, weight, and pain, while the melanocortin-5 receptor has been investigated for its functions in muscle glucose uptake, secretion of oils in the skin, and potential role in anxiety/depression. Currently, there is a need for compounds that block activation of the melanocortin-4 receptor. Such compounds would be useful to increase appetite and may be useful in the treatment of diseases of negative energy balance, such as cachexia associated with cancer, anorexia, and failure to thrive in children etc. Compounds that blocked activation of the melanocortin-4 receptor can also produce an analgesic effect (relief from pain).

There is also a need for compounds that block activation of the melanocortin-5 receptor. Such compounds would be useful to modulate muscle glucose uptake and secretion of oils in the skin. Such compounds would be useful treat anxiety or depression and as probes for investigating the role of the MC5R in its variation functions.

### SUMMARY OF THE INVENTION

Applicant has identified compounds that block activation of the melanocortin-4 receptor. Such compounds are useful to increase appetite and may be useful in the treatment of diseases of negative energy balance such as, e.g., cachexia associated with cancer, anorexia, and failure to thrive in children, etc. Compounds that block activation of the melanocortin-4 receptor can also produce an analgesic effect (relief from pain). Accordingly, in one embodiment, the invention provides a compound of formula (I):

R¹—A¹—A²—A³—A⁴—N(R²)₂ (I)

wherein:

R¹ is H, (C₂-C₆)alkanoyl, (C₂-C₆)cycloalkyl or (C₂-C₄)alkyl, optionally substituted with cycloalkyl;

each R² is independently H or (C₂-C₆)alkyl;

A¹ is a residue of an aromatic D-amino acid;

A² is a residue of a basic amino acid;

A³ is a residue of an aromatic L- or a D-amino acid; and

A⁴ is a residue of an L- or D-amino acid.

By way of example, and not limitation, exemplary compounds of formula (I) may include those shown in FIGS. 16-20. In some embodiments, the compound is Ac-DNal(2′)-Arg-DNal(2′)-DLys-NH₂, Ac-DNal(2′)-Arg-DNal(2′)-Arg-NH₂, Ac-DPhe(pI)-hArg-Nal(2′)-Arg-NH₂, Ac-DPhe(pI)-Arg-Nal(2′)-Om-NH₂ or Ac-DPhe(pI)-Arg-Nal(2′)-Dab-NH₂.

Certain embodiments of the invention provide a pharmaceutical composition comprising a compound of formula (I) as described herein, or a pharmaceutically acceptable salt thereof, and a pharmaceutically acceptable carrier.

Certain embodiments of the invention provide a compound of formula (I) as described herein, or a pharmaceutically acceptable salt thereof, for use in medical therapy.

Certain embodiments of the invention provide a dietary supplement comprising a compound of formula (I) as described herein, or a salt thereof.

Certain embodiments of the invention provide a method for increasing appetite, treating a disease of negative energy balance (e.g., cachexia, anorexia, or failure to thrive in children), or promoting analgesia in a mammal, comprising administering an effective amount of a compound of formula (I) as described herein, or a pharmaceutically acceptable salt thereof, to the mammal.

Certain embodiments of the invention provide a compound of formula (I) as described herein, or a pharmaceutically acceptable salt thereof, for increasing appetite, treating a disease of negative energy balance (e.g., cachexia, anorexia, or failure to thrive in children), or promoting analgesia (e.g. in a mammal).

Certain embodiments of the invention provide the use of a compound of formula (I) as described herein, or a pharmaceutically acceptable salt thereof, to prepare a medicament for increasing appetite, treating a disease of negative energy balance (e.g., cachexia, anorexia, or failure to thrive in children), or promoting analgesia in a mammal.

Applicant has also discovered compounds that block activation of the melanocortin-5 receptor. Such compounds are useful to modulate muscle glucose uptake and secretion of oils in the skin. Such compounds are also useful treat anxiety or depression and as probes for investigating the role of the MC5R in its variation functions. Accordingly, in one embodiment, the invention provides a compound of formula (II):

R³—A⁵—A⁶—A⁷—A⁸—N(R⁴)₂ (II)

wherein:

R³ is H, (C₁-C₆)alkanoyl, (C₁-C₆)cycloalkyl or (C₁-C₄)alkyl, optionally substituted with cycloalkyl;

each R⁴ is independently H or (C₁-C₆)alkyl;

A⁵ is a residue of an aromatic D-amino acid;

A⁶ is a residue of an aromatic D-amino acid;

A⁷ is a residue of an aromatic L- or a D-amino acid; and

A⁸ is a residue of a basic amino acid.

By way of example, and not limitation, exemplary compounds of formula (II) may include those shown in FIGS. 16-18. In some embodiments, the compound is Ac-DNal(2′)-DNal(2′)-DNal(2′)-Arg-NH₂, Ac-DNal(2′)-DNal(2′)-DPhe(pI)-Arg-NH₂, Ac-DPhe(pI)-DNal(2′)-DNal(2′)-Arg-NH₂, Ac-DPhe(pI)-DNal(2′)-DPhe(pI)-Arg-NH₂ or Ac-DPhe(pI)-DNal(2′)-Nal(2′)-Arg-NH₂.

Certain embodiments of the invention provide a pharmaceutical composition comprising a compound of formula (II) as described herein, or a pharmaceutically acceptable salt thereof, and a pharmaceutically acceptable carrier.

Certain embodiments of the invention provide a compound of formula (II) as described herein, or a pharmaceutically acceptable salt thereof, for use in medical therapy.

Certain embodiments of the invention provide a dietary supplement comprising a compound of formula (II) as described herein, or a salt thereof.

Certain embodiments of the invention provide a method for modulating muscle glucose uptake, secretion of oils in the skin, treating acne, or treating anxiety or depression in a mammal, comprising administering an effective amount of a compound of formula (II) as described herein, or a pharmaceutically acceptable salt thereof, to the mammal.

Certain embodiments of the invention provide a compound of formula (II) as described herein, or a pharmaceutically acceptable salt thereof, for modulating muscle glucose uptake, secretion of oils in the skin, treating acne, or treating anxiety or depression (e.g. in a mammal).

Certain embodiments of the invention provide the use of a compound of formula (II) as described herein, or a pharmaceutically acceptable salt thereof, to prepare a medicament for modulating muscle glucose uptake, secretion of oils in the skin, treating acne, or treating anxiety or depression in a mammal.

The invention also provides a compound (e.g. an amino acid sequence) as described herein.

The invention also provides processes and synthetic intermediates described herein that can be used to prepare a compound of formula (I) or formula (II).

### BRIEF DESCRIPTION OF THE FIGURES

FIG. 1. Illustration of the primary antagonist screening results of the TPI924 library at the mMC4R. The TPI924 library (240 mixtures) was screened at 50 μg/mL concentrations in the presence of 0.5 nM NDP-MSH in a cAMP based fluorescent β-galactosidase assay. The fluorescence signal (indicative of cAMP levels), was normalized to well protein levels, plate basal signal, and plate maximal NDP-MSH signal. The normalized signal was converted to a percentage from the median plate experimental value to basal signal. A larger percentage corresponds to more potent putative antagonist activity. The X-axes represent the amino acid residue that was held constant at that position (O) of a tetrapeptide library with the three remaining positions composed of 60 amino acids. Mixtures are arranged by putative antagonist potency. Black dots indicate mixtures that were selected for a follow-up two-point concentration-response assay. Amino acids tested but not shown on the A1 X-axes and having less putative antagonist potency than LPhe(pNO2) include, in order, Phe, Nal(2′), Asp, DPhe, Ser, Asn, Ala(3-Pyr), Ala(2-Thi), Gln, DCha, DAso, DAla, Thr, DGlu, DThr, DHis, βAla, His, DTc, DSer, DIle, Tic, DLys. Amino acids tested but not shown on the A2 X-axes and having less putative antagonist potency than DAla(3-Pyr), in order, Pro, Ala(3-Pyr), Tic, DHis, Leu, βAla, Ala, DSer, DGlu, DPhe(pCl), ε-Aminocaporic acid, Gly, Dphe(pNO2), Met, Phe, Gln, DTyr, Asp, DAsn, Glu, Asn, DAla(2-Thi), DAsp. Amino acids tested but not shown on the A3 X-axes and having less putative antagonist potency than DNle include, in order, DNle, Ala(2-Thi), DHis, Thr, Gly, βAla, Pro, Gln, DAsn, Met[O2], DAla(2-Thi), DMet, ε-Aminocaporic acid, DPhe, Tic, Ser, DTyr, DGlu, DAla, DAsp. Amino acids tested but not shown on the A4 X-axes and having less putative antagonist potency than Leu include, in order,DCha, Ala, Ala(2-Thi), Pro, Phe, ε-Aminocaporic acid, DMet, Asp, Val, Thr, DAla, DPhe(pNO2), Tyr, dehydpPro, Met[O2], Phe(pNO2), DTry, DLeu.

FIG. 2. Illustration of the follow-up two-point antagonist concentration-response screen of the TPI924 library at the mMC4R. The antagonist screen was assayed using 0.5 nM NDP-MSH in the presence of 25 or 50 μg/mL TPI924 concentrations (for each grouping 50 μg/mL TPI924 is shown on the left and 25 μg/mL TPI924 is on the right). The fluorescence signal was normalized to well protein levels, plate basal signal, and plate maximal NDP-MSH signal. The normalized signal was converted to a percentage from the signal of control 0.5 nM NDP-MSH (without mixture) to basal signal. A higher percentage corresponds to more potent putative antagonist activity. The X-axis represents the amino acid residue that was held constant at that position (position indicated below) with the three remaining positions composed of 60 amino acids. Mixtures are grouped by position within the tetrapeptide scaffold and arranged by putative antagonist potency using the 25 μg/mL TPI924 with 0.5 nM NDP-MSH results. Black dots indicate amino acids at specific positions that were used in the library design of individual tetrapeptides.

FIG. 3. Structures of amino acids.

FIG. 4. Illustration of the antagonist pharmacology of 48 (COR1-25) and 9 (COR2-87) at the mMC3R and mMC4R.

FIG. 5. Comparison of mMC4R pA₂ values for paired tetrapeptides. The paired compounds have three residues in common and vary at the indicated position. Compounds 24 (MDE10-28, triangle on the left) and 48 (COR1-25, square on the left) are highlighted with an asterisk in the position 1 plot. Compounds 36 (COR2-3, triangle on the right) and 48 (COR1-25, square on the right) are highlighted with an asterisk in the position 2 plot.

FIG. 6. Illustration of the agonist pharmacology of NDP-MSH, 9 (COR2-87), 42 (COR1-7), 44 (COR1-105), 45 (COR1-13), and 48 (COR1-25) at the mMC1R and mMC5R.

FIG. 7. Plot of HPLC k′ (ACN) Versus mMCR pA₂ Values. Compounds are grouped based upon how many basic charges each tetrapeptide possesses.

FIG. 8. mMC4R SAR Summary. Pie charts representing the number of peptides synthesized with a particular amino acid at the indicated position and the ranges of mMC4R antagonist potency (pA₂). The number of peptides represented within each pie chart out of the forty synthesized and assayed is shown in the lower right of the chart.

FIG. 9. Comparison of mMC4R pA₂ Values Between Paired Tetrapeptides by Position. Paired compounds have three residues in common and vary at the indicated position.

FIG. 10. mMC3R SAR Summary. Pie charts representing the number of peptides synthesized with a particular amino acid at the indicated position and the ranges of mMC3R antagonist potency (pA₂). The number of peptides represented within each pie chart out of the forty synthesized and assayed is shown in the lower right of the chart.

FIG. 11. Comparison of mMC3R pA₂ Values Between Paired Tetrapeptides by Position. Paired compounds have three residues in common and vary at the indicated position.

FIG. 12. mMC3R Versus mMC4R pA₂ Values for Tetrapeptides with the Ac-Aromatic-Basic-Aromatic-Basic-NH₂ Substitution Pattern.

FIG. 13. mMC4R/mMC3R Fold Selectivity Between Paired Tetrapeptides by Position. Paired compounds have three residues in common and vary at the indicated position. The pA₂ values were converted to antagonist Ki [antagonist Ki=10^{-(pA}^{₂}⁾] for comparison.

FIG. 14. Preliminary Agonist and Antagonist Mixture Based Positional Scan Results with Standard Errors. Results for each position are grouped together and are ordered by antagonist mean within each position. Data were initial normalized to plate protein, basal, and maximal NDP-MSH levels. Singleton outlying points were eliminated using the quartile method. An X indicates that the position was selected for the two-concentration follow-up study.

FIG. 15. Two-Concentration Agonist and Antagonist Mixture Based Positional Scan Follow-up Results with Standard Errors. Results for each position are grouped together and are ordered by antagonist mean at the 25 μg/mL concentration within each position. Data were initial normalized to plate protein, basal, and maximal NDP-MSH levels. Singleton outlying points were eliminated using the quartile method. An X indicates that the position was selected for deconvolution and individual compound synthesis.

FIG. 16 shows analytical data for the representative tetrapeptides.

FIG. 17 shows representative tetrapeptide antagonist pharmacology at the mouse melanocortin receptors.

FIG. 18 shows representative tetrapeptide antagonist pharmacology at the mouse melanocortin receptors.

FIG. 19 shows characterization of representative antagonists.

FIG. 20 shows characterization of representative antagonists.

### DETAILED DESCRIPTION

Certain embodiments of the invention provide a compound of formula (I) or (II), or a salt thereof, comprising one or more protecting groups. In certain embodiments, the protecting group is Boc, Fmoc or Tos. In certain embodiments, one or more amino acid side-chains contain a protecting group (e.g., Boc, Fmoc or Tos). In certain embodiments, the protecting group is Tos.

In cases where compounds are sufficiently basic or acidic, a salt of a compound of formula (I) or (II) can be useful as an intermediate for isolating or purifying a compound of formula (I) or (II). Additionally, administration of a compound of formula (I) or (II) as a pharmaceutically acceptable acid or base salt may be appropriate. Examples of pharmaceutically acceptable salts are organic acid addition salts formed with acids which form a physiological acceptable anion, for example, tosylate, methanesulfonate, acetate, citrate, malonate, tartarate, succinate, benzoate, ascorbate, α-ketoglutarate, and α-glycerophosphate. Suitable inorganic salts may also be formed, including hydrochloride, sulfate, nitrate, bicarbonate, and carbonate salts.

Pharmaceutically acceptable salts may be obtained using standard procedures well known in the art, for example by reacting a sufficiently basic compound such as an amine with a suitable acid affording a physiologically acceptable anion. Alkali metal (for example, sodium, potassium or lithium) or alkaline earth metal (for example calcium) salts of carboxylic acids can also be made.

Compounds of formula (I) and (II) (including salts and prodrugs thereof) can be formulated as pharmaceutical compositions and administered to a mammalian host, such as a human patient in a variety of forms adapted to the chosen route of administration, i.e., orally or parenterally, by intravenous, intramuscular, topical, nasal, inhalation, suppository, sub dermal osmotic pump, or subcutaneous routes.

Thus, the present compounds may be systemically administered, e.g., orally, in combination with a pharmaceutically acceptable vehicle such as an inert diluent or an assimilable edible carrier. They may be enclosed in hard or soft shell gelatin capsules, may be compressed into tablets, or may be incorporated directly with the food of the patient's diet. For oral therapeutic administration, the active compound may be combined with one or more excipients and used in the form of ingestible tablets, buccal tablets, troches, capsules, elixirs, suspensions, syrups, wafers, and the like. Such compositions and preparations should contain at least 0.1% of active compound. The percentage of the compositions and preparations may, of course, be varied and may conveniently be between about 2 to about 60% of the weight of a given unit dosage form. The amount of active compound in such therapeutically useful compositions is such that an effective dosage level will be obtained.

The tablets, troches, pills, capsules, and the like may also contain the following: binders such as gum tragacanth, acacia, corn starch or gelatin; excipients such as dicalcium phosphate; a disintegrating agent such as corn starch, potato starch, alginic acid and the like; a lubricant such as magnesium stearate; and a sweetening agent such as sucrose, fructose, lactose or aspartame or a flavoring agent such as peppermint, oil of wintergreen, or cherry flavoring may be added. When the unit dosage form is a capsule, it may contain, in addition to materials of the above type, a liquid carrier, such as a vegetable oil or a polyethylene glycol. Various other materials may be present as coatings or to otherwise modify the physical form of the solid unit dosage form. For instance, tablets, pills, or capsules may be coated with gelatin, wax, shellac or sugar and the like. A syrup or elixir may contain the active compound, sucrose or fructose as a sweetening agent, methyl and propylparabens as preservatives, a dye and flavoring such as cherry or orange flavor. Of course, any material used in preparing any unit dosage form should be pharmaceutically acceptable and substantially non-toxic in the amounts employed. In addition, the active compound may be incorporated into sustained-release preparations and devices.

The active compound may also be administered intravenously or intraperitoneally by infusion or injection. Solutions of the active compound or its salts can be prepared in water, optionally mixed with a nontoxic surfactant. Dispersions can also be prepared in glycerol, liquid polyethylene glycols, triacetin, and mixtures thereof and in oils. Under ordinary conditions of storage and use, these preparations contain a preservative to prevent the growth of microorganisms.

The pharmaceutical dosage forms suitable for injection or infusion can include sterile aqueous solutions or dispersions or sterile powders comprising the active ingredient which are adapted for the extemporaneous preparation of sterile injectable or infusible solutions or dispersions, optionally encapsulated in liposomes. In all cases, the ultimate dosage form should be sterile, fluid and stable under the conditions of manufacture and storage. The liquid carrier or vehicle can be a solvent or liquid dispersion medium comprising, for example, water, ethanol, a polyol (for example, glycerol, propylene glycol, liquid polyethylene glycols, and the like), vegetable oils, nontoxic glyceryl esters, and suitable mixtures thereof. The proper fluidity can be maintained, for example, by the formation of liposomes, by the maintenance of the required particle size in the case of dispersions or by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars, buffers or sodium chloride. Prolonged absorption of the injectable compositions can be brought about by the use in the compositions of agents delaying absorption, for example, aluminum monostearate and gelatin.

Sterile injectable solutions are prepared by incorporating the active compound in the required amount in the appropriate solvent with various of the other ingredients enumerated above, as required, followed by filter sterilization. In the case of sterile powders for the preparation of sterile injectable solutions, the preferred methods of preparation are vacuum drying and the freeze drying techniques, which yield a powder of the active ingredient plus any additional desired ingredient present in the previously sterile-filtered solutions.

For topical administration, the present compounds may be applied in pure form, i.e., when they are liquids. However, it will generally be desirable to administer them to the skin as compositions or formulations, in combination with a dermatologically acceptable carrier, which may be a solid or a liquid.

Useful solid carriers include finely divided solids such as talc, clay, microcrystalline cellulose, silica, alumina and the like. Useful liquid carriers include water, alcohols or glycols or water-alcohol/glycol blends, in which the present compounds can be dissolved or dispersed at effective levels, optionally with the aid of non-toxic surfactants. Adjuvants such as fragrances and additional antimicrobial agents can be added to optimize the properties for a given use. The resultant liquid compositions can be applied from absorbent pads, used to impregnate bandages and other dressings, or sprayed onto the affected area using pump-type or aerosol sprayers.

Thickeners such as synthetic polymers, fatty acids, fatty acid salts and esters, fatty alcohols, modified celluloses or modified mineral materials can also be employed with liquid carriers to form spreadable pastes, gels, ointments, soaps, and the like, for application directly to the skin of the user.

Examples of useful dermatological compositions which can be used to deliver the compounds of formula I or II to the skin are known to the art; for example, see Jacquet et al. (U.S. Pat. No. 4,608,392), Geria (U.S. Pat. No. 4,992,478), Smith et al. (U.S. Pat. No. 4,559,157) and Wortzman (U.S. Pat. No. 4,820,508).

Useful dosages of the compound of formula (I) and (II) can be determined by comparing their in vitro activity, and in vivo activity in animal models. Methods for the extrapolation of effective dosages in mice, and other animals, to humans are known to the art; for example, see U.S. Pat. No. 4,938,949.

The amount of the compound, or an active salt or derivative thereof, required for use in treatment will vary not only with the particular salt selected but also with the route of administration, the nature of the condition being treated and the age and condition of the patient and will be ultimately at the discretion of the attendant physician or clinician.

In general, however, a suitable dose will be in the range of from about 0.5 to about 100 mg/kg, e.g., from about 10 to about 75 mg/kg of body weight per day, such as 3 to about 50 mg per kilogram body weight of the recipient per day, preferably in the range of 6 to 90 mg/kg/day, most preferably in the range of 15 to 60 mg/kg/day.

The compound is conveniently formulated in unit dosage form; for example, containing 5 to 1000 mg, conveniently 10 to 750 mg, most conveniently, 50 to 500 mg of active ingredient per unit dosage form. In one embodiment, the invention provides a composition comprising a compound of the invention formulated in such a unit dosage form.

The desired dose may conveniently be presented in a single dose or as divided doses administered at appropriate intervals, for example, as two, three, four or more sub-doses per day. The sub-dose itself may be further divided, e.g., into a number of discrete loosely spaced administrations; such as multiple inhalations from an insufflator or by application of a plurality of drops into the eye.

The term “amino acid,” comprises the residues of the natural amino acids (e.g. Ala, Arg, Asn, Asp, Cys, Glu, Gln, Gly, His, Hyl, Hyp, Ile, Leu, Lys, Met, Phe, Pro, Ser, Thr, Trp, Tyr, and Val) in D or L form, as well as unnatural amino acids (e.g. PyrAla, ThiAla, (pCl)Phe, (pNO₂)Phe, ε-Aminocaproic acid, Met[O₂], dehydPro, (3I)Tyr, norleucine (Nle), para-I-phenylalanine ((pI)Phe), 2-napthylalanine (2-Nal), β-cyclohexylalanine (Cha), β-alanine (β-Ala), phosphoserine, phosphothreonine, phosphotyrosine, hydroxyproline, gamma-carboxyglutamate; hippuric acid, octahydroindole-2-carboxylic acid, statine, 1,2,3,4,-tetrahydroisoquinoline-3-carboxylic acid (Tic), penicillamine, ornithine (Orn), citrulline (Cit), diaminopimelic acid (Dab), diaminopimelic acid (Dap), homoarginine (hArg), α-methyl-alanine, para-benzoylphenylalanine, phenylglycine, propargylglycine, sarcosine, and tert-butylglycine) in D or L form. The term also comprises natural and unnatural amino acids bearing a conventional amino protecting group (e.g. acetyl or benzyloxycarbonyl), as well as natural and unnatural amino acids protected at the carboxy terminus (e.g. as a (C₁-C₆)alkyl, phenyl or benzyl ester or amide; or as an α-methylbenzyl amide). Other suitable amino and carboxy protecting groups are known to those skilled in the art (See for example, T. W. Greene, Protecting Groups In Organic Synthesis; Wiley: New York, 1981, and references cited therein

The term “aromatic L-amino acid” includes L amino acids having side chain that comprises one or more aromatic rings. For example, the term includes L-Nal(2′), L-Phe(pI), and L-Trp. In one embodiment, the aromatic ring is an aryl group. In one embodiment, the aromatic ring is a heteroaryl ring. In one embodiment, the aromatic ring is a 6-10 membered aryl group. In one embodiment, the aromatic ring is a 5- or 6-menbered heteroaryl ring. In one embodiment, the aromatic ring is a phenyl or naphthyl ring. In one embodiment, the aromatic ring is optionally substituted with one or more groups independently selected from halo.

The term “aromatic D-amino acid” includes D amino acids having side chain that comprises one or more aromatic rings. For example, the term includes D-Nal(2′), D-Phe(pI), and D-Trp.

The term “basic amino acid” includes amino acids having side chain that comprises one or more basic groups. For example, the term includes Lys, Arg, His, Orn, Dab, Dap, hArg, D-Arg, D-Lys, and Cit.

As used herein, the term “residue of an amino acid” means an amino acid wherein one or more atoms (e.g., H or OH) have been removed to provide an open valence that is used to link the amino acid to form a peptide bond or to link to a carboxy-terminal group (e.g., R¹- or R³) or to link an amino terminus to R² or R⁴.

The term “cycloalkyl” refers to a saturated or partially unsaturated (non-aromatic) all carbon ring having 3 to 8 carbon atoms (i.e., (C₃-C₈)carbocycle). The term also includes multiple condensed, saturated all carbon ring systems (e.g., ring systems comprising 2, 3 or 4 carbocyclic rings). Accordingly, carbocycle includes multicyclic carbocyles such as a bicyclic carbocycles (e.g., bicyclic carbocycles having about 3 to 15 carbon atoms, about 6 to 15 carbon atoms, or 6 to 12 carbon atoms such as bicyclo[3.1.0]hexane and bicyclo[2.1.1]hexane), and polycyclic carbocycles (e.g tricyclic and tetracyclic carbocycles with up to about 20 carbon atoms). The rings of the multiple condensed ring system can be connected to each other via fused, spiro and bridged bonds when allowed by valency requirements. For example, multicyclic carbocyles can be connected to each other via a single carbon atom to form a spiro connection (e.g., spiropentane, spiro[4,5]decane, etc), via two adjacent carbon atoms to form a fused connection (e.g., carbocycles such as decahydronaphthalene, norsabinane, norcarane) or via two non-adjacent carbon atoms to form a bridged connection (e.g., norbomane, bicyclo[2.2.2]octane, etc). Non-limiting examples of cycloalkyls include cyclopropyl, cyclobutyl, cyclopentyl, cyclohexyl, bicyclo [2.2.1]heptane, pinane, and adamantane.

The term “aryl” as used herein refers to a single all carbon aromatic ring or a multiple condensed all carbon ring system wherein at least one of the rings is aromatic. For example, in certain embodiments, an aryl group has 6 to 20 carbon atoms, 6 to 14 carbon atoms, 6 to 12 carbon atoms, or 6 to 10 carbon atoms. Aryl includes a phenyl radical. Aryl also includes multiple condensed carbon ring systems (e.g., ring systems comprising 2, 3 or 4 rings) having about 9 to 20 carbon atoms in which at least one ring is aromatic and wherein the other rings may be aromatic or not aromatic (i.e., cycloalkyl. The rings of the multiple condensed ring system can be connected to each other via fused, spiro and bridged bonds when allowed by valency requirements. It is to be understood that the point of attachment of a multiple condensed ring system, as defined above, can be at any position of the ring system including an aromatic or a carbocycle portion of the ring. Non-limiting examples of aryl groups include, but are not limited to, phenyl, indenyl, indanyl, naphthyl, 1, 2, 3, 4-tetrahydronaphthyl, anthracenyl, and the like.

The term “heterocycle” refers to a single saturated or partially unsaturated ring that has at least one atom other than carbon in the ring, wherein the atom is selected from the group consisting of oxygen, nitrogen and sulfur; the term also includes multiple condensed ring systems that have at least one such saturated or partially unsaturated ring, which multiple condensed ring systems are further described below. Thus, the term includes single saturated or partially unsaturated rings (e.g., 3, 4, 5, 6 or 7-membered rings) from about 1 to 6 carbon atoms and from about 1 to 3 heteroatoms selected from the group consisting of oxygen, nitrogen and sulfur in the ring. The sulfur and nitrogen atoms may also be present in their oxidized forms. Exemplary heterocycles include but are not limited to azetidinyl, tetrahydrofuranyl and piperidinyl. The term “heterocycle” also includes multiple condensed ring systems (e.g., ring systems comprising 2, 3 or 4 rings) wherein a single heterocycle ring (as defined above) can be condensed with one or more groups selected from cycloalkyl, aryl, and heterocycle to form the multiple condensed ring system. The rings of the multiple condensed ring system can be connected to each other via fused, spiro and bridged bonds when allowed by valency requirements. It is to be understood that the individual rings of the multiple condensed ring system may be connected in any order relative to one another. It is also to be understood that the point of attachment of a multiple condensed ring system (as defined above for a heterocycle) can be at any position of the multiple condensed ring system including a heterocycle, aryl and carbocycle portion of the ring. In one embodiment the term heterocycle includes a 3-15 membered heterocycle. In one embodiment the term heterocycle includes a 3-10 membered heterocycle. In one embodiment the term heterocycle includes a 3-8 membered heterocycle. In one embodiment the term heterocycle includes a 3-7 membered heterocycle. In one embodiment the term heterocycle includes a 3-6 membered heterocycle. In one embodiment the term heterocycle includes a 4-6 membered heterocycle. In one embodiment the term heterocycle includes a 3-10 membered monocyclic or bicyclic heterocycle comprising 1 to 4 heteroatoms. In one embodiment the term heterocycle includes a 3-8 membered monocyclic or bicyclic heterocycle heterocycle comprising 1 to 3 heteroatoms. In one embodiment the term heterocycle includes a 3-6 membered monocyclic heterocycle comprising 1 to 2 heteroatoms. In one embodiment the term heterocycle includes a 4-6 membered monocyclic heterocycle comprising 1 to 2 heteroatoms. Exemplary heterocycles include, but are not limited to aziridinyl, azetidinyl, pyrrolidinyl, piperidinyl, homopiperidinyl, morpholinyl, thiomorpholinyl, piperazinyl, tetrahydrofuranyl, dihydrooxazolyl, tetrahydropyranyl, tetrahydrothiopyranyl, 1,2,3,4-tetrahydroquinolyl, benzoxazinyl, dihydrooxazolyl, chromanyl, 1,2-dihydropyridinyl, 2,3-dihydrobenzofuranyl, 1,3-benzodioxolyl, 1,4-benzodioxanyl, spiro[cyclopropane-1,1′-isoindolinyl]-3′-one, isoindolinyl-1-one, 2-oxa-6-azaspiro[3.3]heptanyl, imidazolidin-2-one imidazolidine, pyrazolidine, butyrolactam, valerolactam, imidazolidinone, hydantoin, dioxolane, phthalimide, and 1,4-dioxane.

The term “heteroaryl” as used herein refers to a single aromatic ring that has at least one atom other than carbon in the ring, wherein the atom is selected from the group consisting of oxygen, nitrogen and sulfur; “heteroaryl” also includes multiple condensed ring systems that have at least one such aromatic ring, which multiple condensed ring systems are further described below. Thus, “heteroaryl” includes single aromatic rings of from about 1 to 6 carbon atoms and about 1-4 heteroatoms selected from the group consisting of oxygen, nitrogen and sulfur. The sulfur and nitrogen atoms may also be present in an oxidized form provided the ring is aromatic. Exemplary heteroaryl ring systems include but are not limited to pyridyl, pyrimidinyl, oxazolyl or furyl. “Heteroaryl” also includes multiple condensed ring systems (e.g., ring systems comprising 2, 3 or 4 rings) wherein a heteroaryl group, as defined above, is condensed with one or more rings selected from cycloalkyl, aryl, heterocycle, and heteroaryl. It is to be understood that the point of attachment for a heteroaryl or heteroaryl multiple condensed ring system can be at any suitable atom of the heteroaryl or heteroaryl multiple condensed ring system including a carbon atom and a heteroatom (e.g., a nitrogen). Exemplary heteroaryls include but are not limited to pyridyl, pyrrolyl, pyrazinyl, pyrimidinyl, pyridazinyl, pyrazolyl, thienyl, indolyl, imidazolyl, triazolyl, tetrazolyl, oxazolyl, isoxazolyl, thiazolyl, furyl, oxadiazolyl, thiadiazolyl, quinolyl, isoquinolyl, benzothiazolyl, benzoxazolyl, indazolyl, quinoxalyl, and quinazolyl.

The terms “treat” and “treatment” refer to both therapeutic treatment and prophylactic or preventative measures, wherein the object is to prevent or decrease an undesired physiological change or disorder, such as a metabolic disorder (e.g., obesity or cachexia) or a disease associated with the metabolic disorder. For purposes of this invention, beneficial or desired clinical results include, but are not limited to, alleviation of symptoms, diminishment of extent of disease, stabilized (i.e., not worsening) state of disease, delay or slowing of disease progression, amelioration or palliation of the disease state, and remission (whether partial or total), whether detectable or undetectable. “Treatment” can also mean prolonging survival as compared to expected survival if not receiving treatment. Those in need of treatment include those already with the condition or disorder as well as those prone to have the condition or disorder or those in which the condition or disorder is to be prevented.

The term “mammal” as used herein refers to, e.g., humans, higher non-human primates, rodents, domestic, cows, horses, pigs, sheep, dogs and cats. In one embodiment, the mammal is a human. In one embodiment, the mammal is a female human. In one embodiment, the mammal is a male human.

### EXEMPLARY EMBODIMENTS

1. A compound of formula (I):

R¹—A¹—A²—A³—A⁴13 N(R²)₂ (I)

wherein:

R¹ is H, (C₁-C₆)alkanoyl, (C₂-C₆)cycloalkyl or (C₂-C₄)alkyl, optionally substituted with cycloalkyl;

each R² is independently H or (C₂-C₆)alkyl;

A¹ is a residue of an aromatic D-amino acid;

A² is a residue of a basic amino acid;

A³ is a residue of an aromatic L- or a D-amino acid; and

A⁴ is a residue of an L- or D-amino acid.
2. The compound of embodiment 1, wherein A¹ is a residue of D-Nal(2′), D-Phe(pI), or D-Trp.
3. The compound of embodiment 1, wherein A¹ is a residue of D-Nal(2′).
4. The compound of embodiment 1, wherein A¹ is a residue of D-Phe(pI).
5. The compound of embodiment 1, wherein A¹ is a residue of D-Trp.
6. The compound of any one of embodiments 1-5, wherein A² is Arg, His, Lys, Orn, Dab, Dap, hArg, D-Arg, D-Lys, or Cit.
7. The compound of any one of embodiments 1-5, wherein A² is Arg.
8. The compound of any one of embodiments 1-5, wherein A² is His.
9. The compound of any one of embodiments 1-5, wherein A² is Lys.
10. The compound of any one of embodiments 1-5, wherein A² is Orn.
11. The compound of any one of embodiments 1-5, wherein A² is Dab.
12. The compound of any one of embodiments 1-5, wherein A² is Dap.
13. The compound of any one of embodiments 1-5, wherein A² is hArg.
14. The compound of any one of embodiments 1-5, wherein A² is D-Arg.
15. The compound of any one of embodiments 1-5, wherein A² is D-Lys.
16. The compound of any one of embodiments 1-5, wherein A² is Cit.
17. The compound of any one of embodiments 1-16, wherein A³ is a residue of D-Nal(2′), D-Phe(pI), or D-Trp.
18. The compound of any one of embodiments 1-16, wherein A³ is a residue of D-Nal(2′).
19. The compound of any one of embodiments 1-16, wherein A³ is a residue of D-Phe(pI).
20. The compound of any one of embodiments 1-16, wherein A³ is a residue of D-Trp.
21. The compound of any one of embodiments 1-16, wherein A³ is a residue of L-Nal(2′), L-Phe(pI), or L-Trp.
22. The compound of any one of embodiments 1-16, wherein A³ is a residue of L-Nal(2′).
23. The compound of any one of embodiments 1-16, wherein A³ is a residue of L-Phe(pI).
24. The compound of any one of embodiments 1-16, wherein A³ is a residue of L-Trp.
25. The compound of any one of embodiments 1-24, wherein A⁴ is a residue of a basic amino acid.
26. The compound of any one of embodiments 1-25, wherein A⁴ is Arg, His, Lys, Orn, Dab, Dap, hArg, D-Arg, D-Lys, or Cit.
27. The compound of any one of embodiments 1-25, wherein A⁴ is Arg.
28. The compound of any one of embodiments 1-25, wherein A⁴ is His.
29. The compound of any one of embodiments 1-25, wherein A⁴ is Lys.
30. The compound of any one of embodiments 1-25, wherein A⁴ is Orn.
31. The compound of any one of embodiments 1-25, wherein A⁴ is Dab.
32. The compound of any one of embodiments 1-25, wherein A⁴ is Dap.
33. The compound of any one of embodiments 1-25, wherein A⁴ is hArg.
34. The compound of any one of embodiments 1-25, wherein A⁴ is D-Arg.
35. The compound of any one of embodiments 1-25, wherein A⁴ is D-Lys.
36. The compound of any one of embodiments 1-25, wherein A⁴ is Cit.
37. The compound of embodiment 1 selected from the group consisting of:

Ac-DNal(2′)-Arg-DNal(2′)-DLys-NH₂

Ac-DNal(2′)-Arg-DNal(2′)-Arg-NH₂

Ac-DNal(2′)-Arg-Trp-DLys-NH₂

Ac-DNal(2′)-Arg-Trp-Arg-NH₂

Ac-DNal(2′)-Arg-DPhe(pI)-DLys-NH₂

Ac-DNal(2′)-Arg-DPhe(pI)-Arg-NH₂

Ac-DNal(2′)-Arg-Nal(2′)-DLys-NH₂

Ac-DNal(2′)-Arg-Nal(2′)-Arg-NH₂

Ac-DPhe(pI)-Arg-DNal(2′)-DLys-NH₂

Ac-DPhe(pI)-Arg-DNal(2′)-Arg-NH₂

Ac-DPhe(pI)-Arg-Trp-DLys-NH₂

Ac-DPhe(pI)-Arg-Trp-Arg-NH₂

Ac-DPhe(pI)-Arg-DPhe(pI)-DLys-NH₂

Ac-DPhe(pI)-Arg-DPhe(pI)-Arg-NH₂

Ac-DPhe(pI)-Arg-Nal(2′)-DLys-NH₂

Ac-DPhe(pI)-Arg-Nal(2′)-Arg-NH₂
38. The compound of embodiment 1 selected from the group consisting of:

Ac-DPhe(pI)-His-Nal(2′)-Arg-NH₂

Ac-DPhe(pI)-Lys-Nal(2′)-Arg-NH₂

Ac-DPhe(pI)-Orn-Nal(2′)-Arg-NH₂

Ac-DPhe(pI)-Dab-Nal(2′)-Arg-NH₂

Ac-DPhe(pI)-Dap-Nal(2′)-Arg-NH₂

Ac-DPhe(PI)-hArg-Nal(2′)-Arg-NH₂

Ac-DPhe(pI)-DArg-Nal(2′)-Arg-NH₂

Ac-DPhe(pI)-DLys-Nal(2′)-Arg-NH₂

Ac-DPhe(pI)-Cit-Nal(2′)-Arg-NH₂

Ac-DPhe(pI)-Arg-Nal(2′)-His-NH₂

Ac-DPhe(pI)-Arg-Nal(2′)-Lys-NH₂

Ac-DPhe(pI)-Arg-Nal(2′)-Orn-NH₂

Ac-DPhe(pI)-Arg-Nal(2′)-Dab-NH₂

Ac-DPhe(pI)-Arg-Nal(2′)-Dap-NH₂

Ac-DPhe(pI)-Arg-Nal(2′)-hArg-NH₂

Ac-DPhe(pI)-Arg-Nal(2′)-DArg-NH₂

Ac-DPhe(pI)-Arg-Nal(2′)-DLys-NH₂

Ac-DPhe(pI)-Arg-Nal(2′)-Cit-NH₂
39. The compound of embodiment 1 which is Ac-DPhe(pI)-Arg-Nal(2′)-Arg-NH₂.
40. The compound of embodiment 1 which is Ac-DNal(2′)-Arg-DNal(2′)-DLys-NH₂.
41. The compound of embodiment 1 which is Ac-DNal(2′)-Arg-DNal(2′)-Arg-NH₂.
42. The compound of embodiment 1 which is Ac-DPhe(pI)-hArg-Nal(2′)Arg-NH₂.
43. The compound of embodiment 1 which is Ac-DPhe(pI)-Arg-Nal(2′)-Orn-NH₂.
44. The compound of embodiment 1 which is Ac-DPhe(pI)-Arg-Nal(2′)-Dab-NH₂.
45. A pharmaceutical composition comprising a compound of formula (I) as described in any one of claims 1-44 or a pharmaceutically acceptable salt thereof, and a pharmaceutically acceptable carrier.
46. A compound of formula (I) as described in any one of claims 1-44 or a pharmaceutically acceptable salt thereof, for use in medical therapy.
47. A dietary supplement comprising a compound of formula (I) as described in any one of claims 1-44 or a pharmaceutically acceptable salt thereof.
48. A method for increasing appetite, treating a disease of negative energy balance (e.g., cachexia, anorexia, or failure to thrive in children), or promoting analgesia in a mammal, comprising administering an effective amount of a compound of formula (I) as described in any one of claims 1-44 or a pharmaceutically acceptable salt thereof, to the mammal.
49. A compound of formula (I) as described in any one of claims 1-44 or a pharmaceutically acceptable salt thereof, for increasing appetite, treating a disease of negative energy balance, or promoting analgesia (e.g. in a mammal).
50. The use of a compound of formula (I) as described in any one of claims 1-44 or a pharmaceutically acceptable salt thereof, to prepare a medicament for increasing appetite, treating a disease of negative energy balance (e.g., cachexia, anorexia, or failure to thrive in children), or promoting analgesia in a mammal.
51. A compound of formula (II):

R³—A⁵—A⁶—A⁷—A⁸—N(R⁴)₂ (II)

wherein:

R³ is H, (C₁-C₆)alkanoyl, (C₁-C₆)cycloalkyl or (C₁-C₄)alkyl, optionally substituted with cycloalkyl;

each R⁴ is independently H or (C₁-C₆)alkyl;

A⁵ is a residue of an aromatic D-amino acid;

A⁶ is a residue of an aromatic D-amino acid;

A⁷ is a residue of an aromatic L- or a D-amino acid; and

A⁸ is a residue of a basic amino acid.
52. The compound of embodiment 51, wherein A⁵ is a residue of D-Nal(2′), D-Phe(pI), or D-Trp.
53. The compound of embodiment 51, wherein A⁵ is a residue of D-Nal(2′).
54. The compound of embodiment 51, wherein A⁵ is a residue of D-Phe(pI).
55. The compound of embodiment 51, wherein A⁵ is a residue of D-Trp.
56. The compound of any one of embodiments 51-55, wherein A⁶ is a residue of D-Nal(2′), D-Phe(pI), or D-Trp.
57. The compound of any one of embodiments 51-55, wherein A⁶ is a residue of D-Nal(2′).
58. The compound of any one of embodiments 51-55, wherein A⁶ is a residue of D-Phe(pI).
59. The compound of any one of embodiments 51-55, wherein A⁶ is a residue of D-Trp.
60. The compound of any one of embodiments 51-59, wherein A⁷ is a residue of D-Nal(2′), D-Phe(pI), D-Trp, L-Nal(2′), L-Phe(pI), or L-Trp.
61. The compound of any one of embodiments 51-59, wherein A⁷ is a residue of D-Nal(2′).
62. The compound of any one of embodiments 51-59, wherein A⁷ is a residue of D-Phe(pI).
63. The compound of any one of embodiments 51-59, wherein A⁷ is a residue of D-Trp.
64. The compound of any one of embodiments 51-59, wherein A⁷ is a residue of L-Nal(2′).
65. The compound of any one of embodiments 51-59, wherein A⁷ is a residue of L-Phe(pI).
66. The compound of any one of embodiments 51-59, wherein A⁷ is a residue of L-Trp.
67. The compound of any one of embodiments 51-59, wherein A⁸ is a residue of Arg or DLys.
68. The compound of embodiment 51 selected from the group consisting of:

Ac-DNal(2′)-DNal(2′)-DNal(2′)-DLys-NH₂

Ac-DNal(2′)-DNal(2′)-DNal(2′)-Arg-NH₂

Ac-DNal(2′)-DNal(2′)-Trp-DLys-NH₂

Ac-DNal(2′)-DNal(2′)-Trp-Arg-NH₂

Ac-DNal(2′)-DNal(2′)-DPhe(pI)-DLys-NH₂

Ac-DNal(2′)-DNal(2′)-DPhe(pI)-Arg-NH₂

Ac-DNal(2′)-DNal(2′)-Nal(2′)-DLys-NH₂

Ac-DNal(2′)-DNal(2′)-Nal(2′)-Arg-NH₂

Ac-DPhe(pI)-DNal(2′)-DNal(2′)-DLys-NH₂

Ac-DPhe(pI)-DNal(2′)-DNal(2′)-Arg-NH₂

Ac-DPhe(pI)-DNal(2′)-Trp-DLys-NH₂

Ac-DPhe(pI)-DNal(2′)-Trp-Arg-NH₂

Ac-DPhe(pI)-DNal(2′)-DPhe(pI)-DLys-NH₂

Ac-DPhe(pI)-DNal(2′)-DPhe(pI)-Arg-NH₂

Ac-DPhe(pI)-DNal(2′)-Nal(2′)-DLys-NH₂
69. The compound of embodiment 51 which is Ac-DNal(2′)-DNal(2′)-DNal(2′)-Arg-NH₂.
70. The compound of embodiment 51 which is Ac-DNal(2′)-DNal(2′)-DPhe(pI)-Arg-NH₂.
71. The compound of embodiment 51 which is Ac-DPhe(pI)-DNal(2′)-DNal(2′)-Arg-NH₂.
72. The compound of embodiment 51 which is Ac-DPhe(pI)-DNal(2′)-DPhe(pI)-Arg-NH₂.
73. The compound of embodiment 51 which is Ac-DPhe(pI)-DNal(2′)-Nal(2′)-Arg-NH₂.
74. A pharmaceutical composition comprising a compound of formula (II) as described in any one of embodiments 51-73 or a pharmaceutically acceptable salt thereof, and a pharmaceutically acceptable carrier.
75. A compound of formula (II) as described in any one of embodiments 51-73 or a pharmaceutically acceptable salt thereof, for use in medical therapy.
76. A dietary supplement comprising a compound of formula (II) as described in any one of embodiments 51-73 or a pharmaceutically acceptable salt thereof.
77. A method for modulating muscle glucose uptake, secretion of oils in the skin, treating acne, or treating anxiety or depression in a mammal, comprising administering an effective amount of a compound of formula (II) as described in any one of embodiments 51-73 or a pharmaceutically acceptable salt thereof, to the mammal.
78. A compound of formula (II) as described in any one of embodiments 51-73 or a pharmaceutically acceptable salt thereof, for modulating muscle glucose uptake, secretion of oils in the skin, treating acne, or treating anxiety or depression (e.g. in a mammal).
79. The use of a compound of formula (II) as described in any one of embodiments 51-73 or a pharmaceutically acceptable salt thereof, to prepare a medicament for modulating muscle glucose uptake, secretion of oils in the skin, treating acne, or treating anxiety or depression in a mammal.

### EXAMPLES

The invention will now be illustrated by the following non-limiting Examples.

### Example 1

### Identification of Melanocortin-4 Antagonists

To identify novel MC4R peptide antagonists, a mixture-based positional scan was performed using a tetrapeptide library at the mouse (m)MC4R. The mouse versus the human MC4R was selected so that novel peptides discovered herein could be used in the future to directly correlate with in vivo mouse experimental physiological data without the need for additional in vitro experiments. Sixty individual building blocks were incorporated at each position, resulting in 240 mixtures (containing 12,960,000 tetrapeptides) assayed at the mMC4R using the synthetic peptide NDP-MSH as the agonis. Following the initial screen at 50 pg/mL concentrations, the mixtures that resulted in the highest putative antagonist potency were re-screened at concentrations of 25 and 50 pg/mL. Based upon these data, substitutions for each of the four tetrapeptide positions were selected, resulting in the deconvolution and synthesis of a forty-eight-member library. Forty tetrapeptides were soluble in solvents compatible with RP-HPLC and were subsequently assayed for agonist activity at the mMC1R, mMC3R, mMC4R, and mMC5R, as well as for antagonist activity at the mMC3R and mMC4R. The remaining eight tetrapeptides, compromised of aromatic amino acids at each position, were insoluble as anticipated, e.g., Ac-DNal(2′)-DNal(2′)-DNal(2′)-DNal(2′)-NH₂.

### Results & Discussion

### Primary Mixture-Based Positional Screen

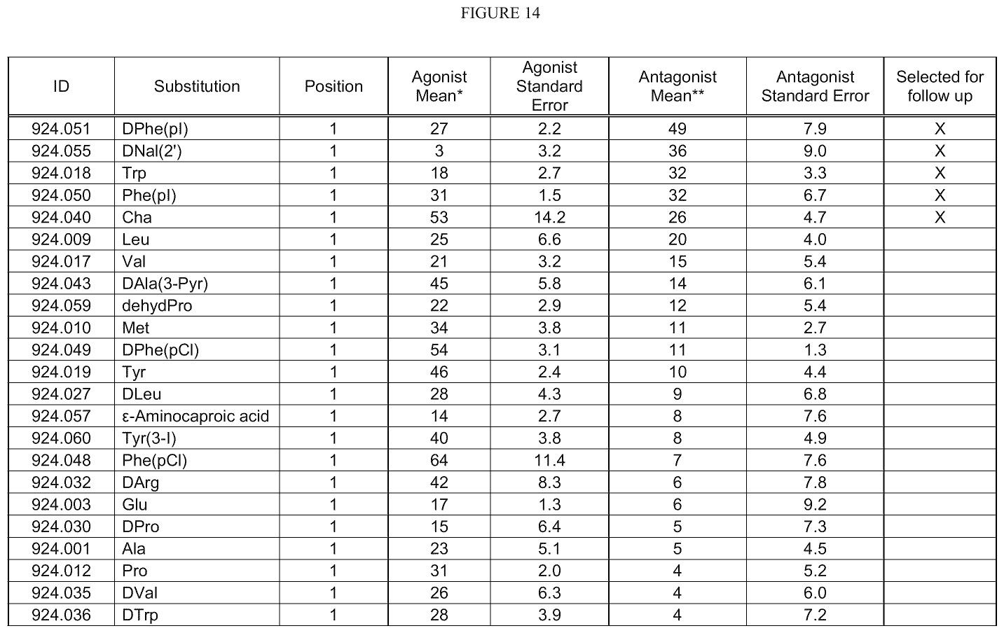

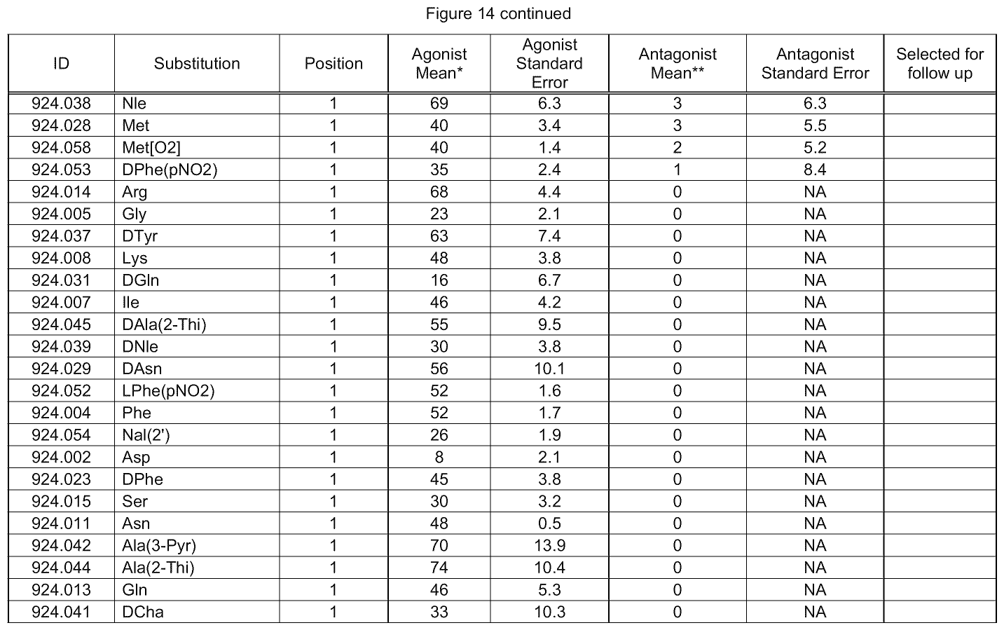

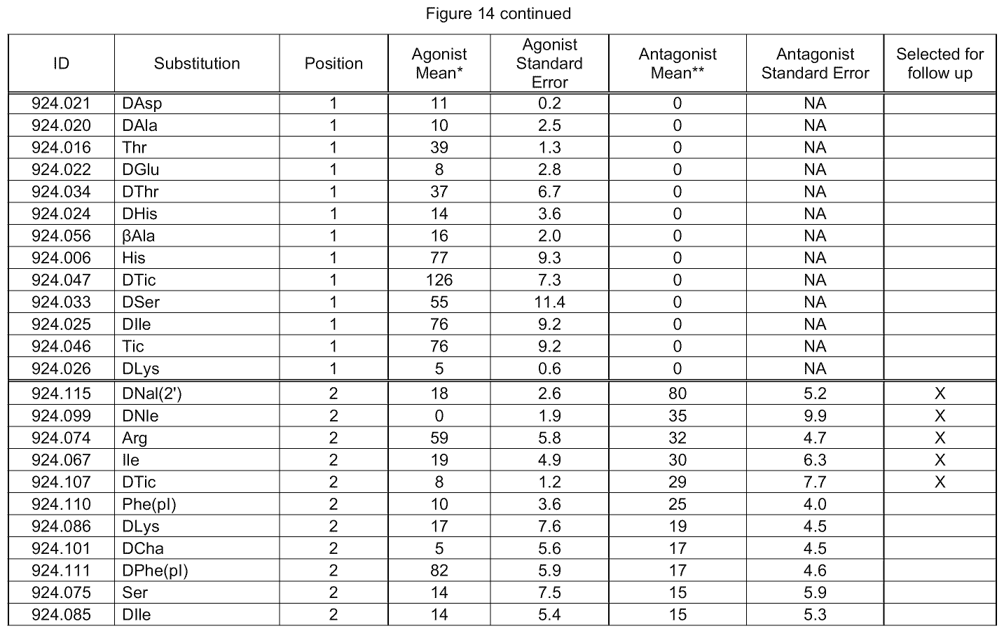

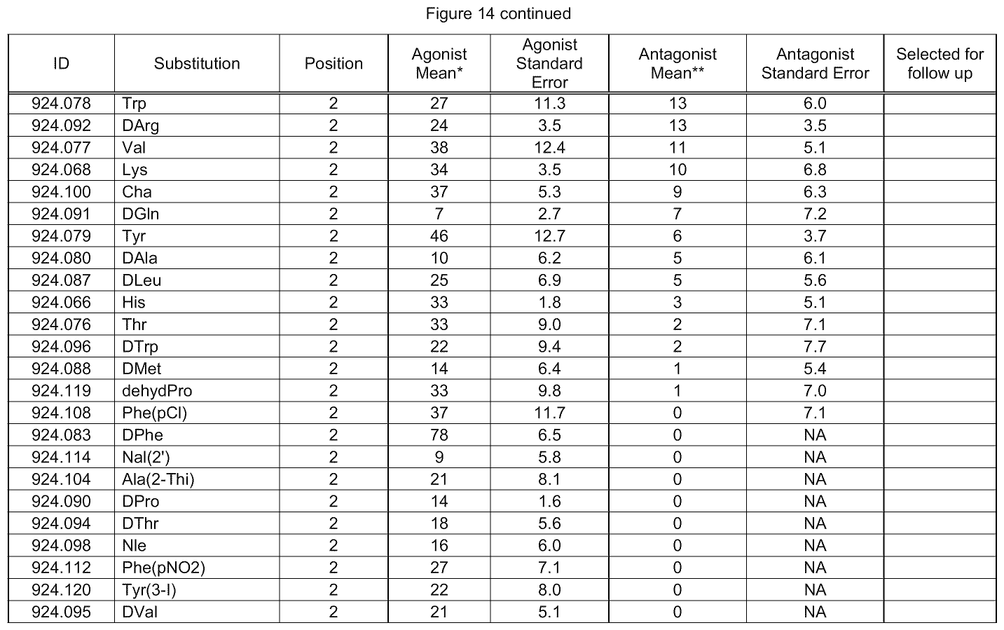

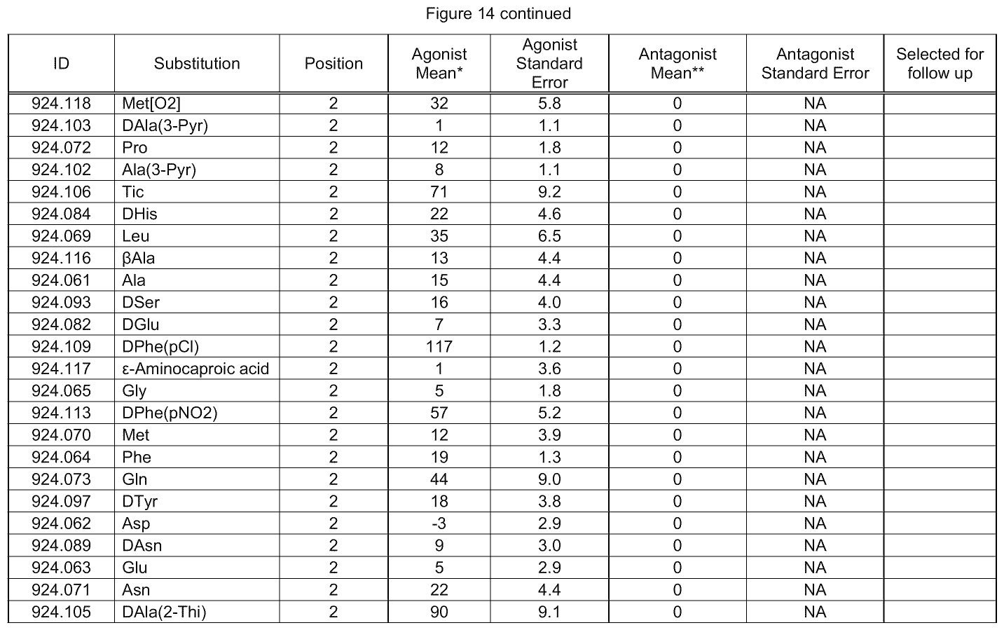

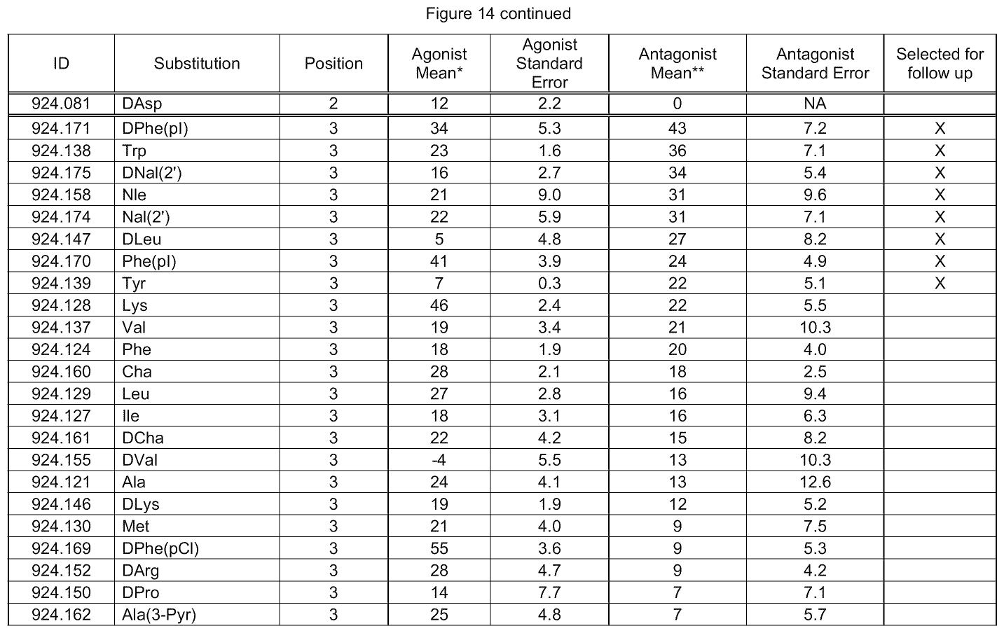

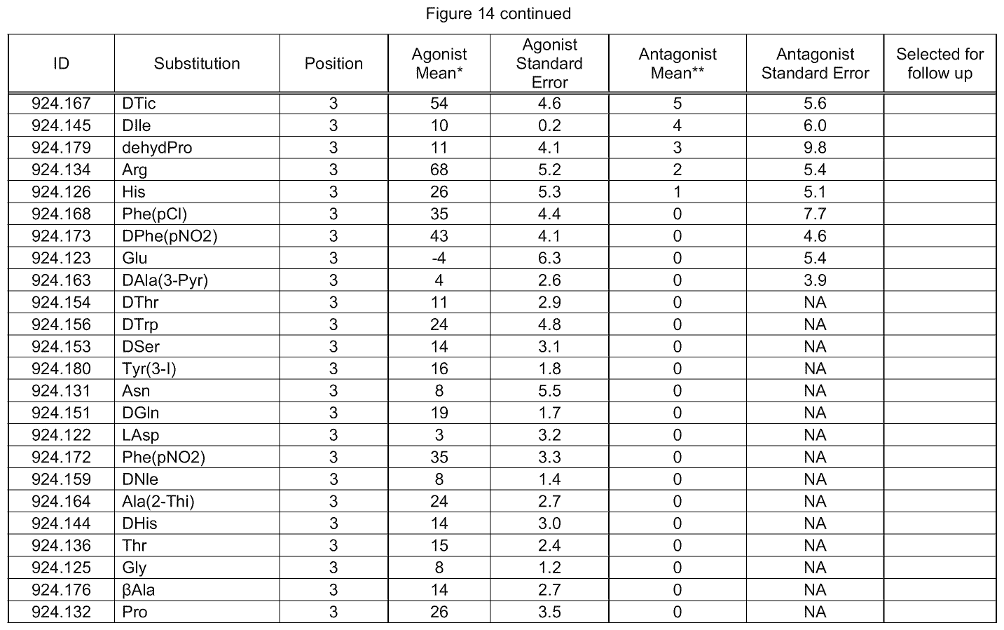

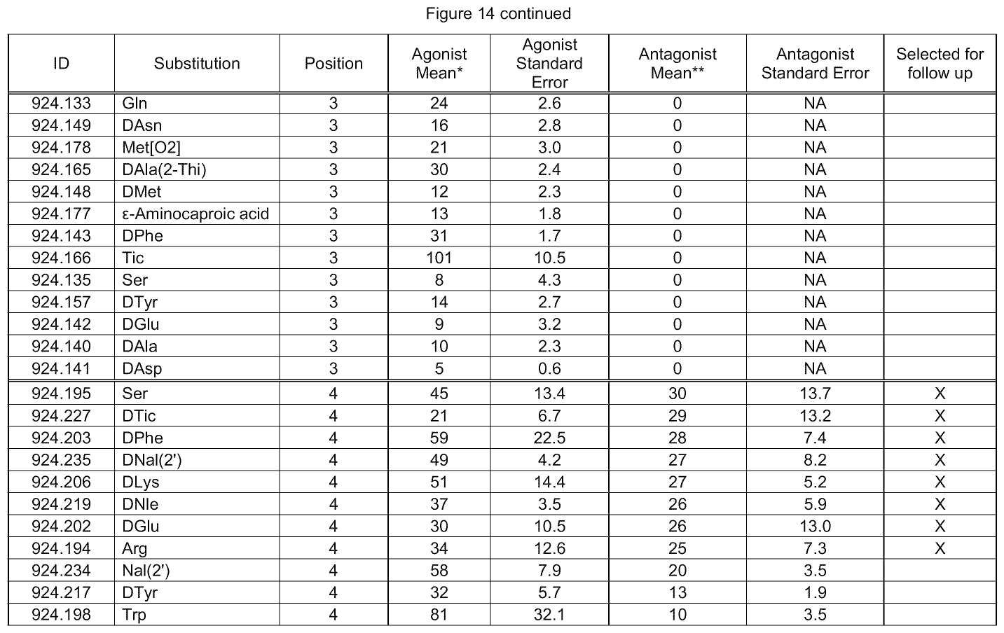

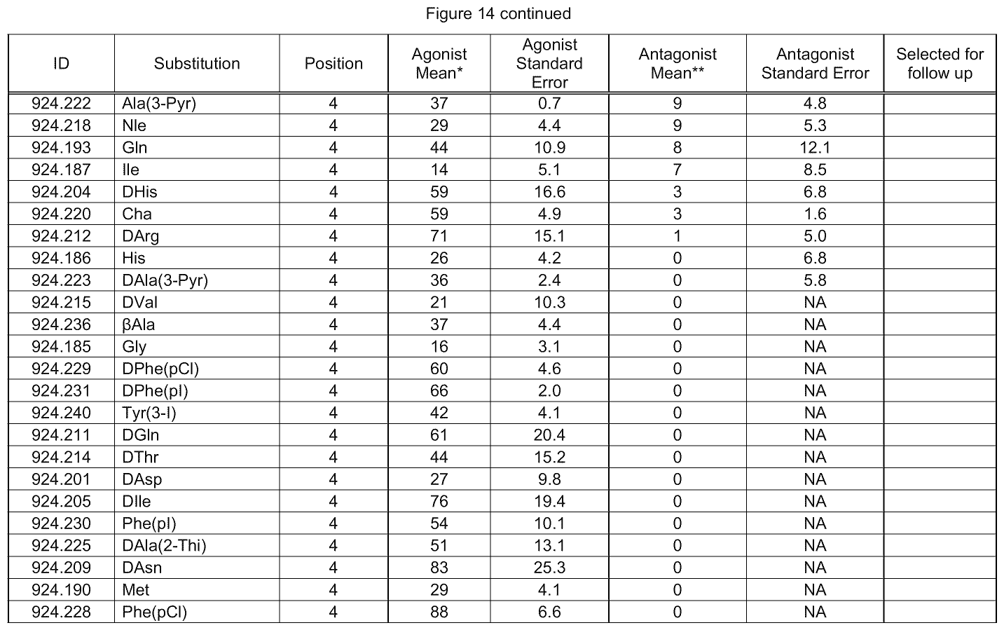

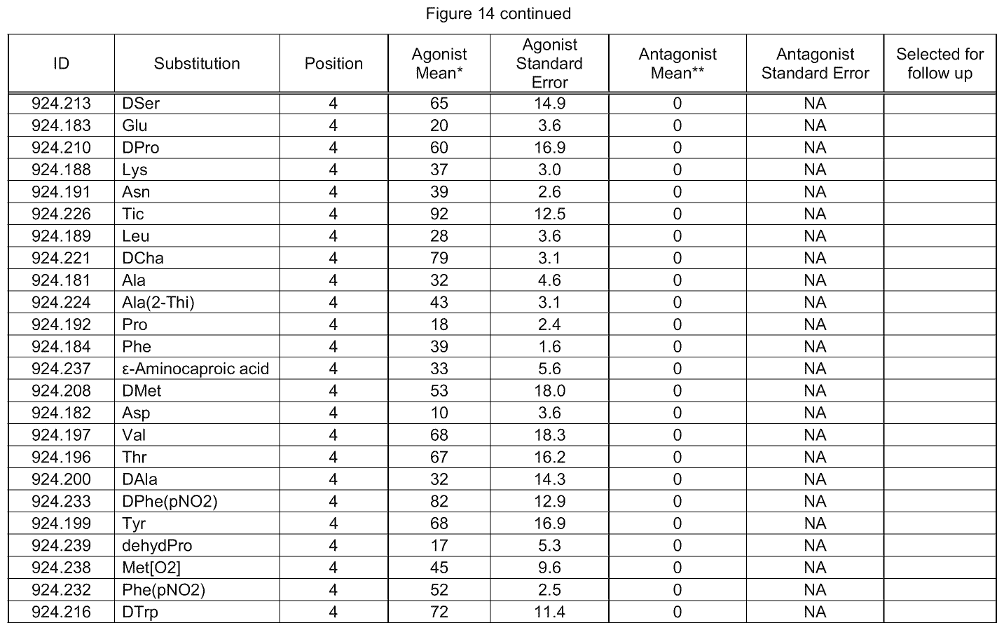

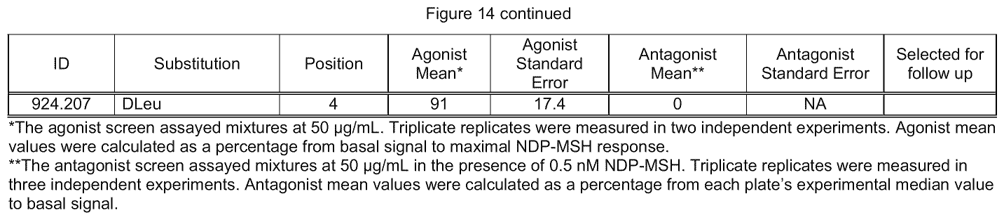

The mixture-based tetrapeptide positional scanning library TPI924, possessing an N-terminal acetyl group and C-terminal amide, was constructed using standard Boc chemistry and the previously reported tea-bag method for generating compound mixtures (Houghten, R. A., Proc. Natl. Acad. Sci. U S. A. 1985, 82 (15), 5131-5135). Each mixture within the library held one position constant while varying the other three positions with sixty natural and unnatural amino acids, resulting in 216,000 (1×60×60×60) tetrapeptides per mixture. The overall library consisted of 12,960,000 (60×60×60×60) tetrapeptides in 240 mixtures, which has previously been used to identify MC3R and MC4R agonist ligands (Haslach, E. M., et al., J. Med. Chem. 2014, 57 (11), 4615-4628; Doering, S. R., et al., J. Med. Chem. 2017, 60 (10), 4342-4357; and Fleming, K. A., et al., J. Med. Chem. 2019, 62 (5), 2738-2749). As an initial screen, the 240 mixtures were assayed in both agonist and antagonist paradigms using a modified β-galactosidase assay (Chen, W. B., et al., Anal. Biochem. 1995, 226 1, 349-354) with the fluorescent substrate 4-methylumbelliferyl-β-D-galactopyranoside to measure cAMP production in HEK293 cells stably expressing the mMC4R. The agonist screen utilized a mixture concentration of 50 μg/mL to identify which mixtures could induce an increase in cAMP in cells expressing the mMC4R (FIG. 14). The established positive control tetrapeptide Ac-His-DPhe-Arg-Trp-NH₂ possesses nanomolar agonist potency at the mMC4R (Haskell-Luevano, et al., J. Med. Chem. 2001, 44 (13), 2247-2252). Mixtures possessing this tetrapeptide sequence (His at position A1, DPhe at position A2, Arg at position A3, and Trp at position A4) resulted in a significant agonist response (68-81% compared to the maximal NDP-MSH signal, FIG. 14) for each of these four mixtures, as anticipated, indicating the screen possessed the requisite sensitivity to identify mMC4R agonist sequences.

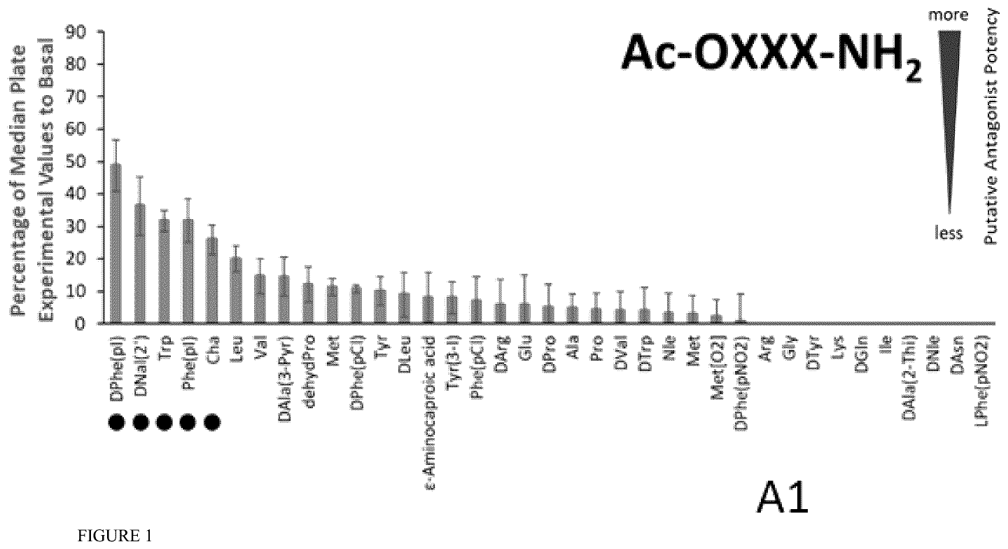

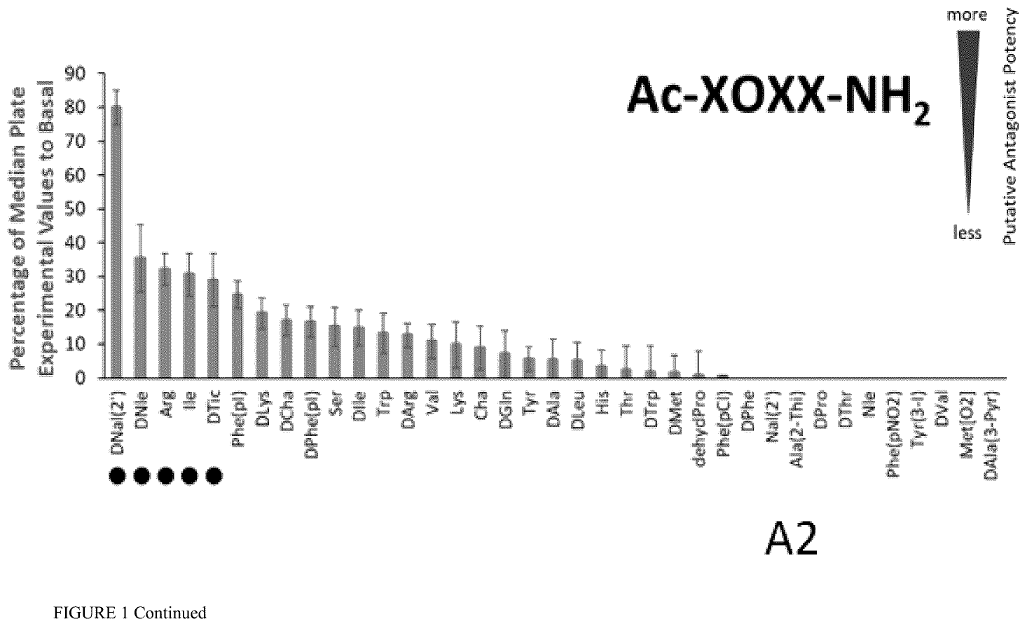

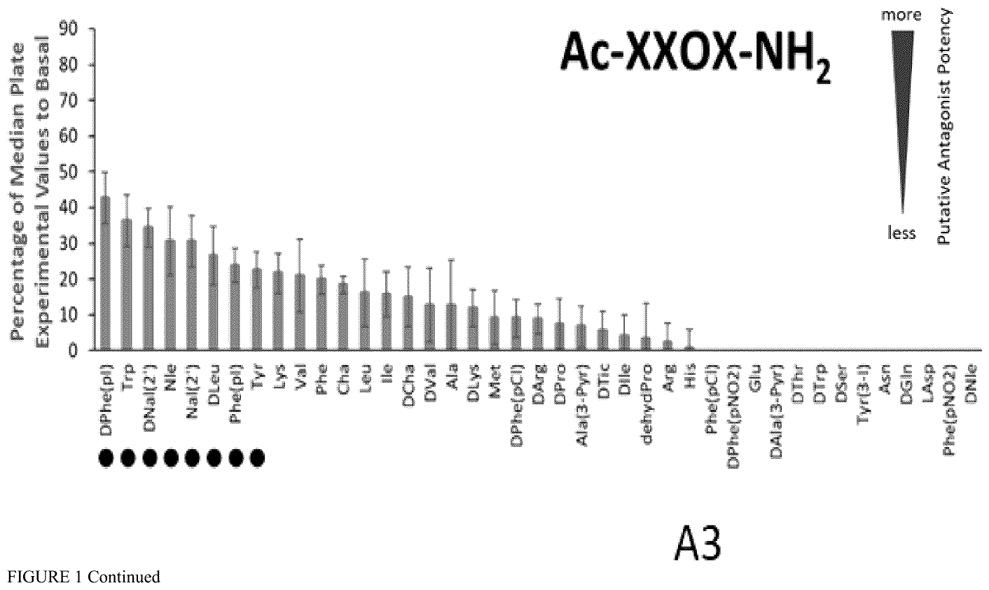

The 240 mixtures were also screened in an antagonist paradigm at a single concentration (50 μg/mL) in the presence of 0.5 nM NDP-MSH (FIG. 1 & FIG. 14). Each well fluorescent signal was normalized to well protein level (cell number control), plate basal signal (negative control), and plate maximal NDP-MSH signal (positive control). The normalized signal was converted to a percentage from the plate median experimental value to basal signal (FIG. 1 & FIG. 14) as an approximation for putative mixture antagonist potency. For each position, the mixtures are arranged in order of observed putative antagonist activity at 50 μg/mL concentrations (FIG. 1). The five to eight mixtures that possessed the highest putative antagonist potency at each position (black dots in FIG. 1, marked as selected for follow-up in FIG. 14) were chosen for rescreening using two concentrations to confirm putative antagonist activity and identify the most active mixtures.

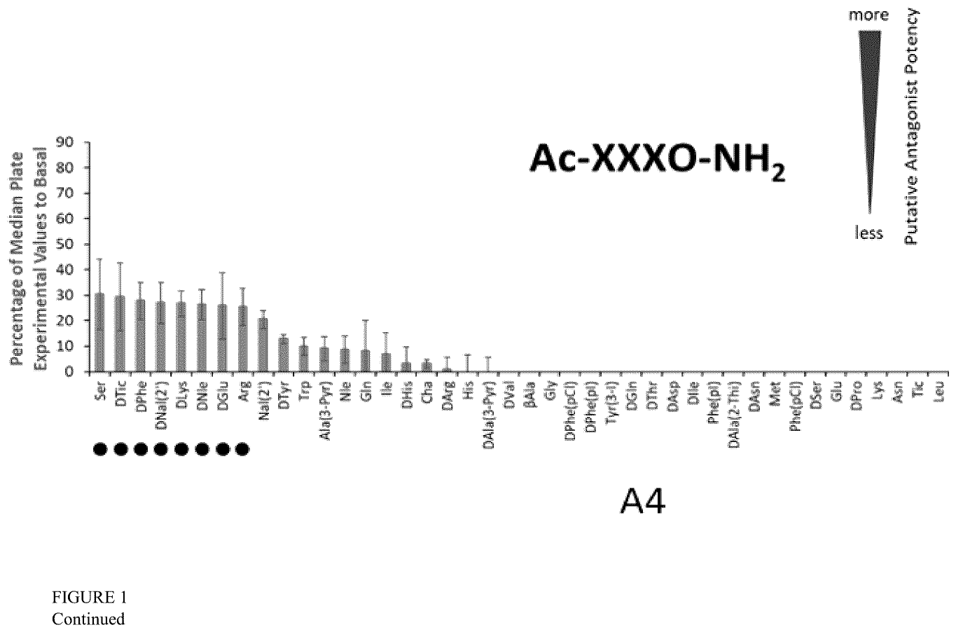

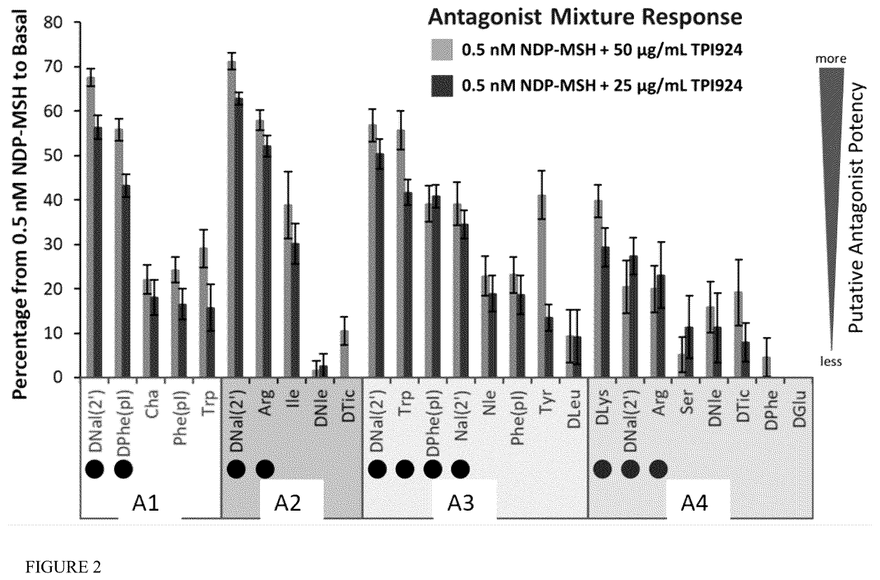

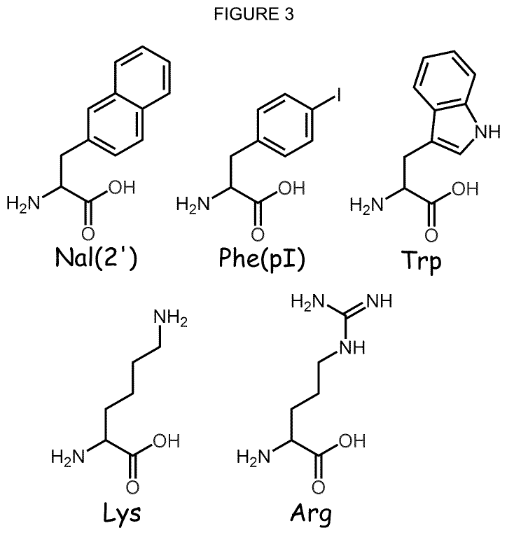

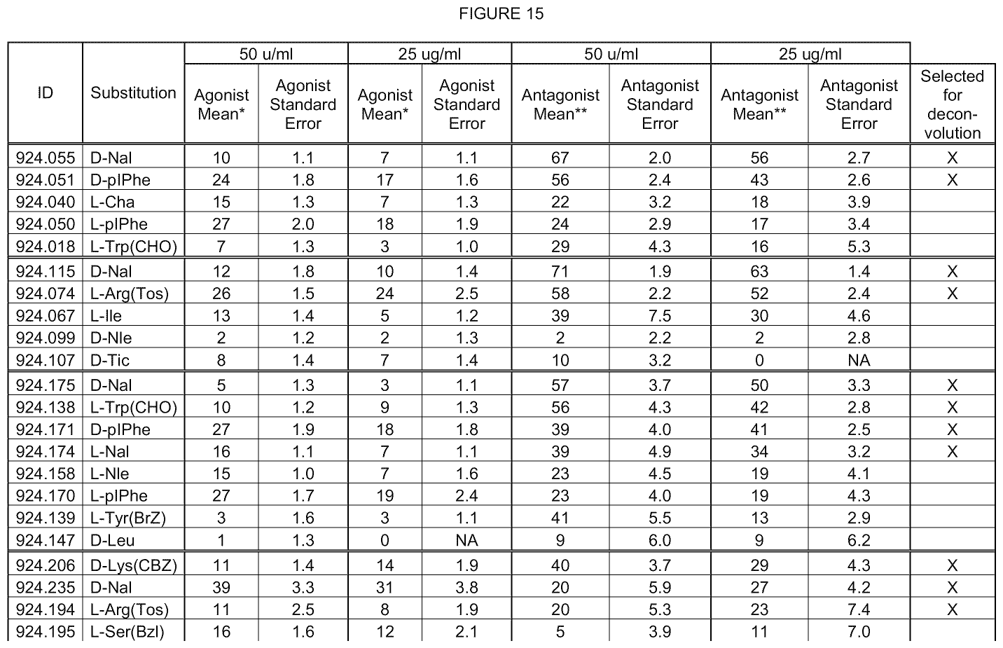

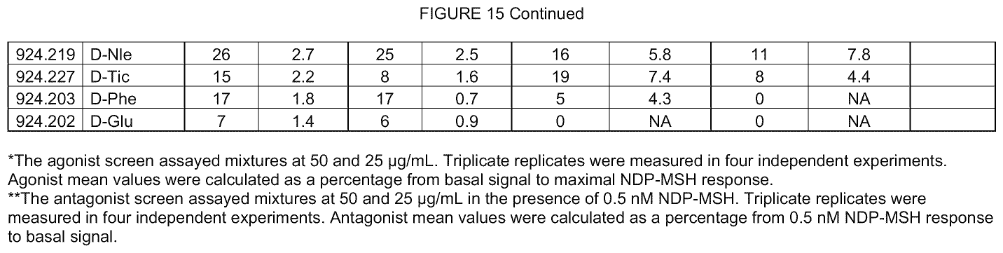

The follow-up screen was performed using twenty-six compound mixtures at two concentrations (25 and 50 μg/mL) in both an agonist (mixture by itself) and antagonist (mixture with 0.5 nM NDP-MSH) paradigm (FIG. 2 and FIG. 15). In the agonist assays, all twenty-six mixtures resulted in less than 40% activity compared to the maximal response of NDP-MSH at the highest 50 pg/mL concentration assayed (FIG. 15). For the antagonist screen, following normalization of the fluorescent signal to protein, basal, and maximal NDP-MSH, the signal for each mixture was converted to a percentage from a control (no mixture) 0.5 nM NDP-MSH signal to basal activity (FIG. 2). Within this approach, larger values indicate more potent putative antagonist activity. Both the 50 and 25 μg/mL mixture concentrations in the presence of 0.5 nM NDP-MSH are presented in FIG. 2, with the mixtures group by position and arranged in observed putative antagonist potency at the 25 μg/mL concentration. Select mixtures did not result in putative antagonism in the follow-up screen (DNle and DTic at the A² position, DPhe and DGlu at the A⁴ position). Of the remaining twenty-two mixtures, seventeen [DNal(2′), DPhe(pI), Cha, Phe(pI), and Trp at A¹, DNal(2′), Arg, and Ile at A², DNal(2′), Trp, Nal(2′), Nle, Phe(pI), and Tyr at A³, and DLys, DNle, and DTic at A⁴] resulted in a concentration-dependent decrease in the putative antagonist signal [the observed antagonist signal was stronger at the 50 μg/mL concentration as compared to the 25 μg/mL concentration], indicating that the putative antagonist signals were dose dependent. The defined amino acids of the mixtures showing the most potent putative antagonist activity at the 25 pg/mL concentration for each position were selected for individual compound synthesis, indicated by black dots on FIG. 2 and summarized in Table 1 and FIG. 3. Individual compounds may be classified into one of four general amino acid substitution patterns: Ac-Aromatic-Aromatic-Aromatic-Aromatic-NH₂, Ac-Aromatic-Basic-Aromatic-Aromatic-NH₂, Ac-Aromatic-Aromatic-Aromatic-Basic-NH₂, and Ac-Aromatic-Basic-Aromatic-Basic-NH₂.

TABLE 1
Summary of Proposed Tetrapeptide Residues Following
Mixture-Based Positional Scanning Deconvolution

| A¹ | A² | A³ | A⁴ |
|---|---|---|---|
| DNal(2') | DNal(2') | DNal(2') | DLys |
| DPhe(pI) | Arg | Trp | DNal(2') |
| | | DPhe(pI) | Arg |
| | | Nal(2') | |

### Individual Compound Library

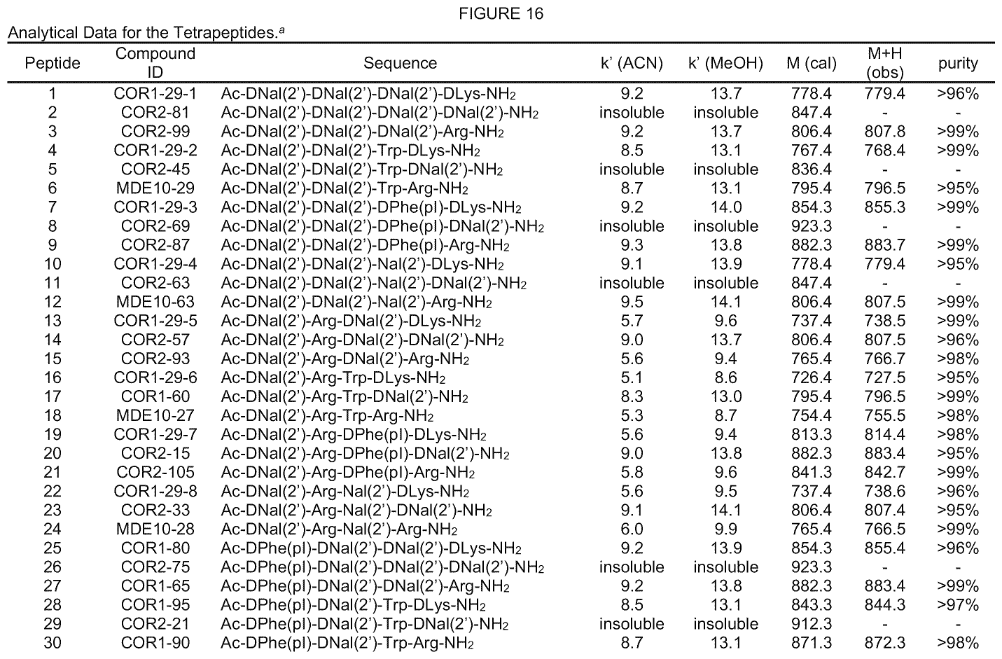

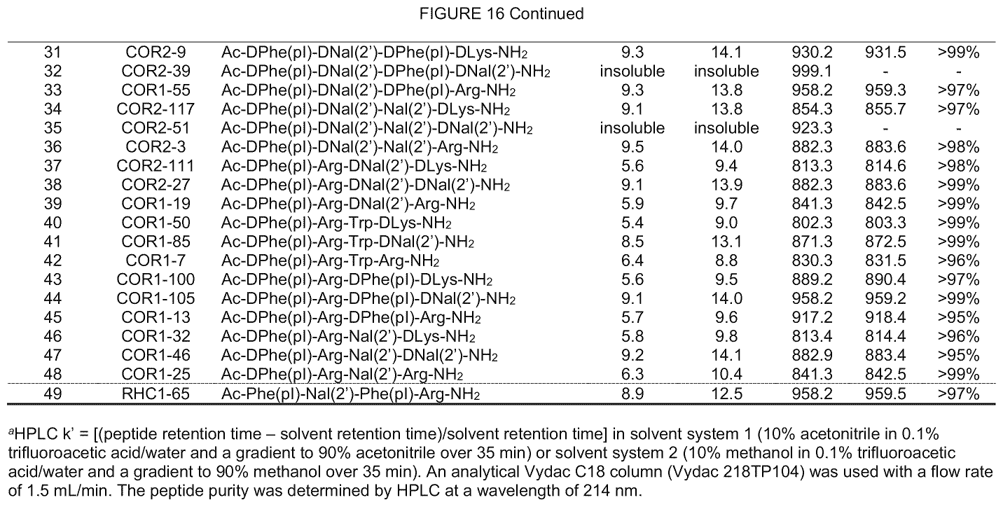

Individual tetrapeptides (FIG. 16) were synthesized manually using standard Fmoc chemical techniques (Carpino, L. A., et al., J. Am. Chem. Soc. 1970, 92 (19), 5748-5749; and Carpino, L. A., et al., J. Org. Chem. 1972, 37 (22), 3404-3409). All tetrapeptides were acetylated at the N-terminal and possessed a C-terminal carboxamide functionality. Following cleavage and sidechain deprotection, peptides were purified by semi-preparative RP-HPLC to greater than 95%. Eight tetrapeptides that were composed of four aromatic amino acids at each position (Ac-Aromatic-Aromatic-Aromatic-Aromatic-NH₂) were not soluble in solvents compatible with RP-HPLC and were not further investigated (FIG. 16, denoted as insoluble). For the remaining forty tetrapeptides, purity was assessed using analytical RP-HPLC and the molecular mass was determined by ESI-MS. The tetrapeptides were assessed for biological activity using the AlphaScreen cAMP assay at the mMC1R, mMC3R, mMC4R, and mMC5R stably expressed in HEK293 cells, as previously described (Singh, A., et al., ACS Med Chem. Lett. 2015, 6 (5), 568-572; Ericson, M. D., et al., Bioorg. Med. Chem. Lett. 2015, 25 (22), 5306-5308; and Lensing, C. J., et al., ACS Chem. Neurosci. 2017, 8 (6), 1262-1278). Since the MC2R is only stimulated by ACTH, it was excluded from this study. Tetrapeptides were assayed for antagonist activity at the mMC4R using NDP-MSH as the agonist, as well as for agonist activity at the mMC1R, mMC3R, and mMC5R. Since none of the compounds were full agonists at the mMC3R at concentrations up to 100 μM, the forty soluble tetrapeptides were also assayed for antagonist activity at the mMC3R with the agonist NDP-MSH. Tetrapeptides that did not possess agonist or antagonist activity in two independent experimental replicates were not further characterized. Compounds were assayed in duplicate wells in at least three independent experiments if agonist or antagonist activity was observed. Tetrapeptides within a 3-fold potency range were considered equipotent due to the inherent error of the assay in our hands. Compounds that activated the receptor to 90% of the maximal signal of NDP-MSH were considered full agonists, while compounds that activated the receptor to less than 20% were considered inactive. Since the cAMP AlphaScreen assay is a loss-of-signal competition assay, in which higher concentrations of compound result in lower assay signal, the data is normalized to baseline and maximal NDP-MSH signal for illustrative purposes, as previously described (Lensing, C. J., et al., J. Med. Chem. 2016, 59 (7), 3112-3128; and Elster, L., et al., J. Biomol. Screening 2007,12 (1), 41-49).

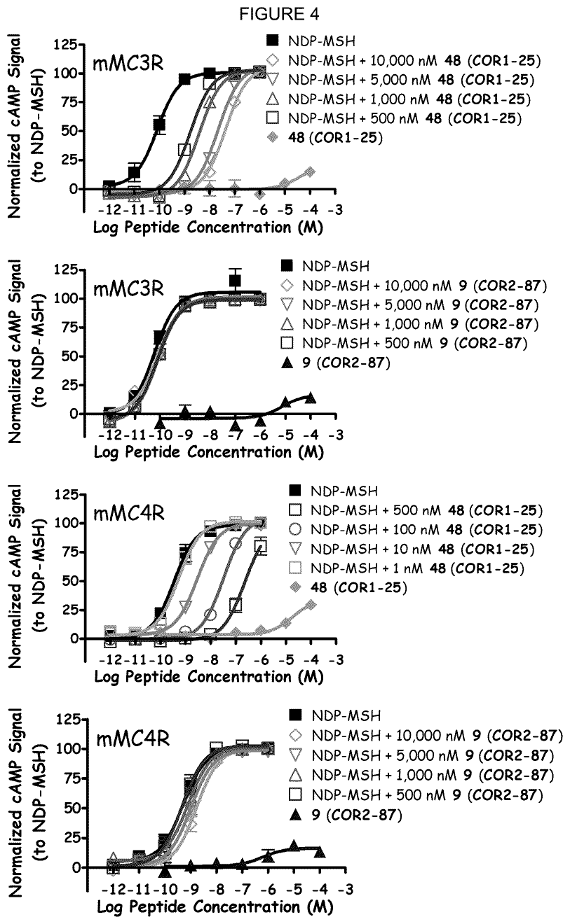

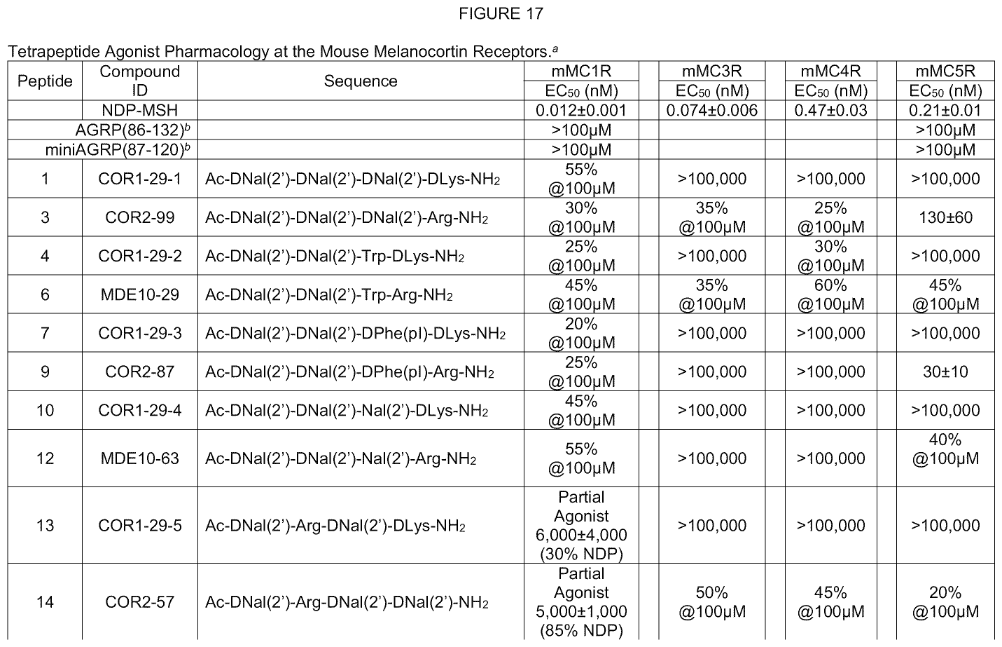

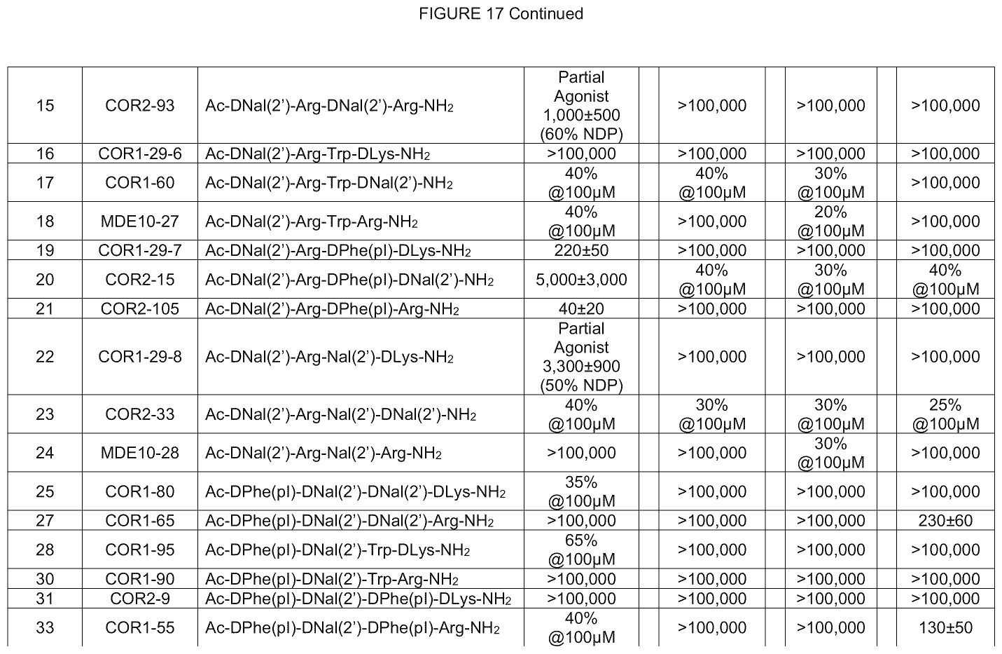

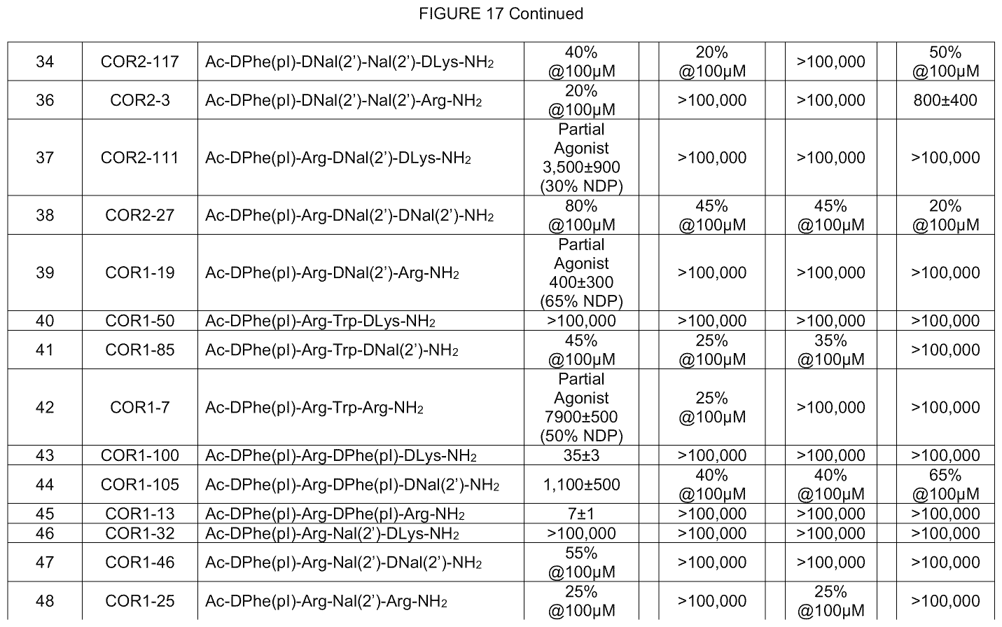

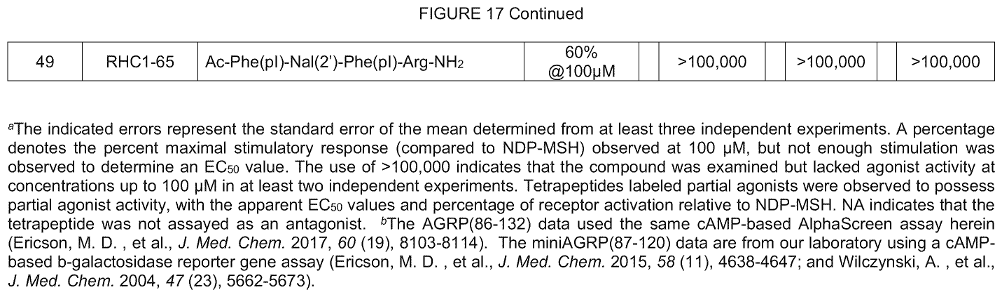

Melanocortin-4 Receptor Activity: The mixtures used to identify the compounds synthesized were screened for both agonist and antagonist activity and the deconvolution was carried out for antagonistic activity. Of the forty tetrapeptides that were purified and screened, none were able to fully stimulate the mMC4R at the highest concentration assayed (100 μM, FIG. 17), an expected result for minimal mMC4R agonist activity based upon the initial mixture screening and the selected amino acids for deconvolution. Thirteen of the forty tetrapeptides were able to partially activate the mMC4R at the highest concentration assayed (100 μM), with signals ranging from 20% to 60% of the maximal signal observed for NDP-MSH. An example of the partial activation is provided in FIG. 4 for 48 (COR1-25), which stimulated the mMC4R to 25% of the maximal NDP-MSH signal at 100 μM concentrations.

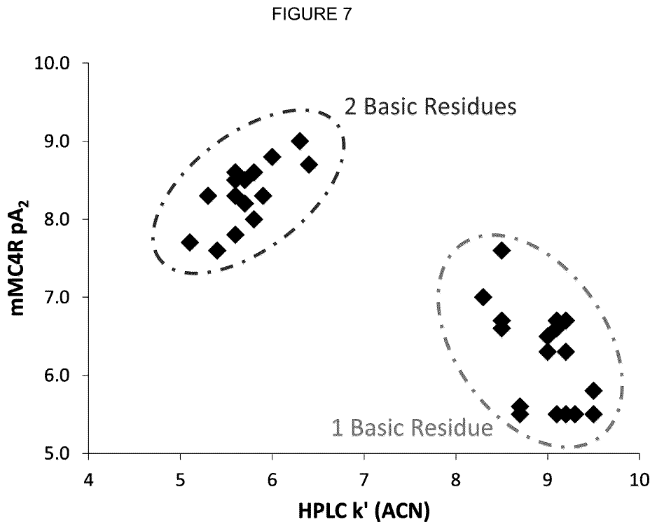

While minimal agonist activity was observed at the mMC4R, thirteen compounds were nanomolar potent antagonists (pA₂ values between 8-9) at the mMC4R (Table 3). All thirteen tetrapeptides possessed the general substitution pattern Ac-Aromatic-Basic-Aromatic-Basic-NH₂. The three remaining tetrapeptides with this substitution pattern, 16 (COR1-29-6), 22 (COR1-29-8), and 40 (COR1-50) possessed pA₂ values of 7.7, 7.8, and 7.6 respectively. Only one compound outside of this substitution pattern that had a pA₂ value greater than 7 [41 (COR1-85), pA₂=7.6]. Overall the Ac-Aromatic-Basic-Aromatic-Basic-NH₂ scaffold yielded the fifteen most potent mMC4R antagonist tetrapeptides in this study. As this scaffold possesses two basic residues compared to one basic residue in either the Ac-Aromatic-Basic-Aromatic-Aromatic-NH₂ or Ac-Aromatic-Aromatic-Aromatic-Basic-NH₂ substitution patterns, the more potent mMC4R antagonists may also be more hydrophilic. Using the HPLC k′ (ACN) value as an approximation for hydrophilicity (smaller value=more hydrophilic), plotting the HPLC k′ (ACN) versus mMC4R pA₂ values results in two distinct compound groups (FIG. 7). The Ac-Aromatic-Basic-Aromatic-Basic-NH₂ pattern groups as a more potent, more hydrophilic cluster, while the single basic substitution patterns possess longer retention times (more hydrophobic) and lower mMC4R antagonist potencies.

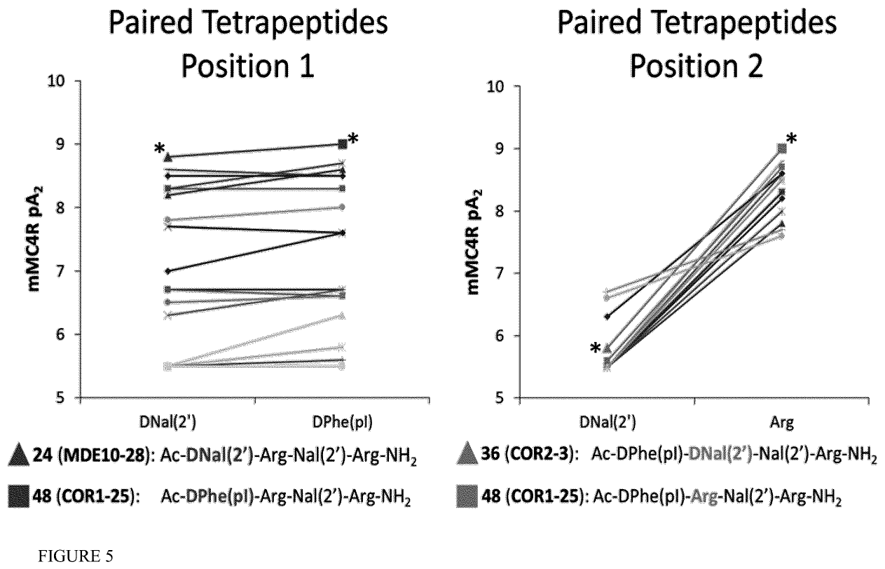

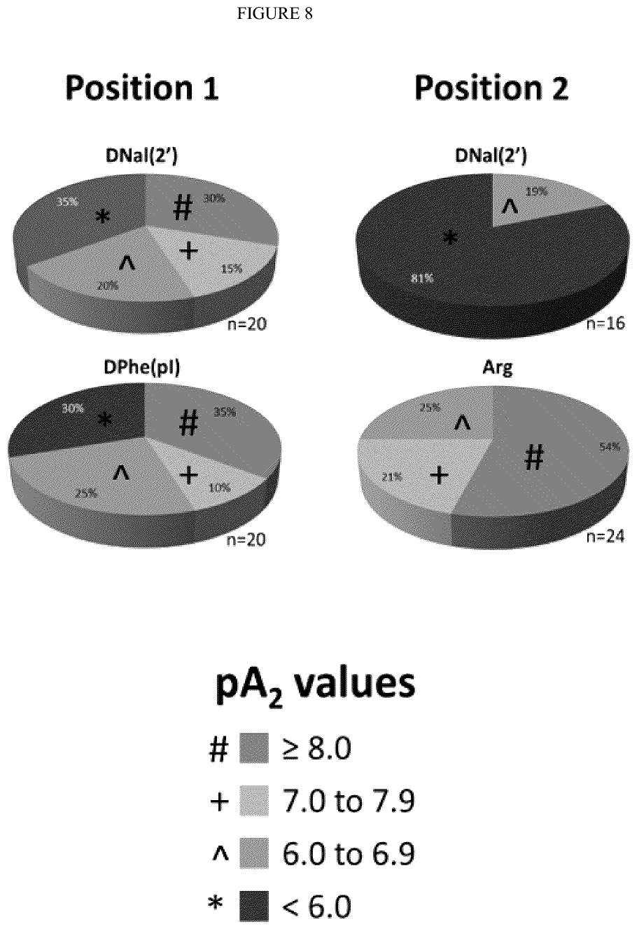

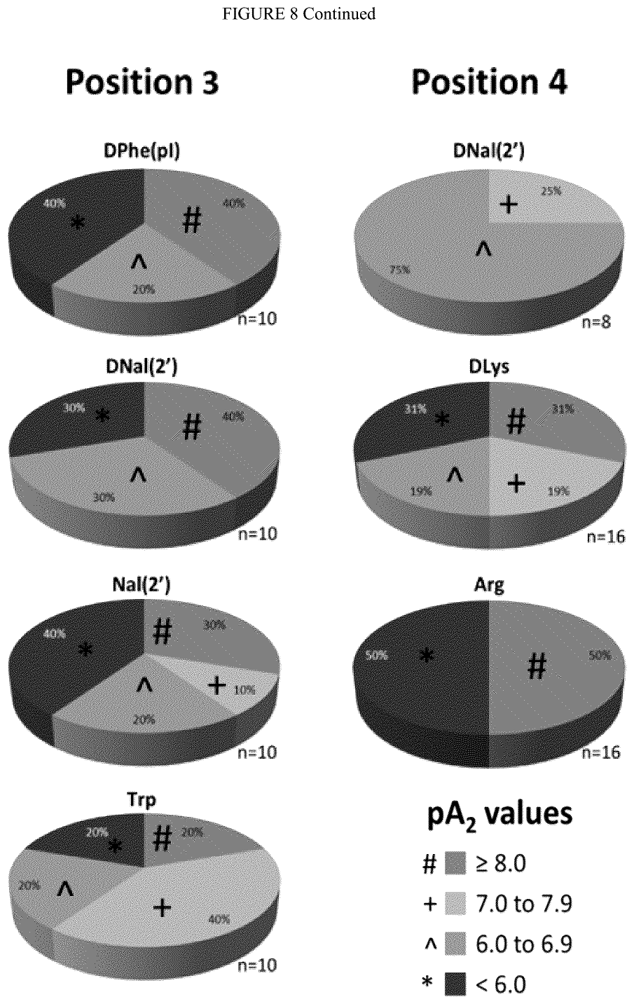

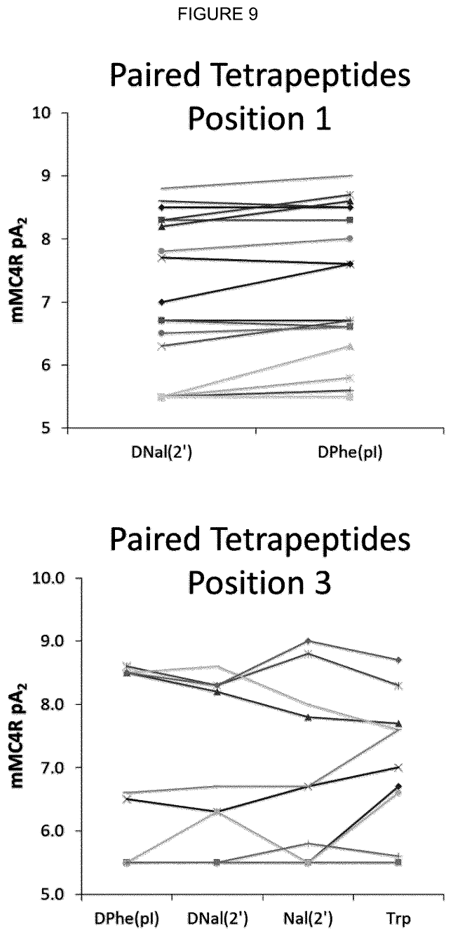

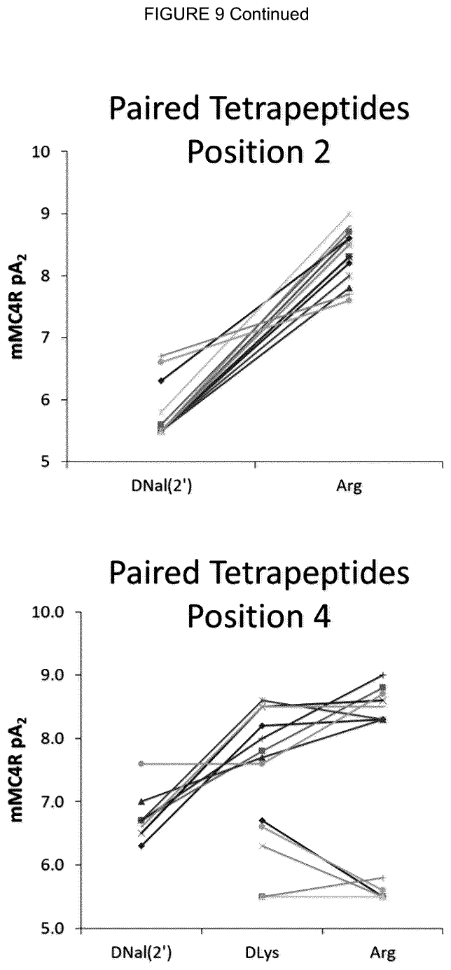

The importance of the basic residues in position 2 and 4 at the mMC4R can be visualized as pie charts comparing the amino acid substitutions within a position with their ranges of pA₂ values (FIG. 8). All the tetrapeptides possessing a DNal(2′) substitution in position 2 resulted in mMC4R antagonist pA₂ values of less than 7 (denoted by ^ and *), while an Arg substitution in position 2 resulted in eighteen tetrapeptides with pA₂ values greater than 7 (denoted by + and #). A similar pattern at position 4 is observed, where eight of sixteen compounds with either the Arg or DLys substitution possess pA₂ values greater than 7 (denoted by + and #), while only two of eight compounds with the DNal residue have a pA₂ between 7 and 7.9 (denoted by +). Another way to visualize the importance of the basic charges is to compare the mMC4R pA₂ values between paired tetrapeptides, in which three of the four amino acids remain constant while the fourth position is varied (FIG. 5 and FIG. 9). The difference in mMC4R pA₂ values between DNal(2′) and DPhe(pI) remains relatively flat a position 1 (FIG. 5), indicating that both substitutions result in similar mMC4R antagonist potency. In contrast, there is a marked trend for increased pA₂ values when Arg is substituted at position 2 compared to DNal(2′), highlighted by compounds 36 (COR2-3) and 48 (COR1-25) in the right panel with an asterisk in FIG. 5. Every Arg substituted tetrapeptide at position 2 possesses a higher mMC4R pA₂ value compared the corresponding DNal(2′) substitution, indicating the importance of Arg at this position. A similar trend is observed at position 4 (FIG. 9), with DNal(2′) substitutions possessing lower mMC4R pA₂ values than the corresponding DLys and Arg containing tetrapeptides, while no apparent trends were observed at position 3.

The most potent tetrapeptide antagonist at the mMC4R, 48, (COR1-25) [Ac-DPhe(pI)-Arg-Nal(2′)-Arg-NH₂], possessed a pA₂ value of 9.0 (Table 3 and FIG. 4). Previous work with longer and more structurally complex compounds such as AGRP, AGRP-derived macrocycles, and the synthetic SHU9119 cyclic peptide with the AlphaScreen Assay resulted in similar pA₂ values at the mMC4R (8.7-9.5) (Ericson, M. D., et al., J. Med. Chem. 2015, 58 (11), 4638-4647; Fleming, K. A., et al., J. Med. Chem. 2018, 61 (17), 7729-7740; Ericson, M. D., et al., J. Med. Chem. 2017, 60 (19), 8103-8114; Ericson, M. D., et al., ACS Chem. Neurosci. 2021,12 (3), 542-556; and Tala, S. R., et al., Bioorg. Med. Chem. Lett. 2015, 25 (24), 5708-5711). The mixture based-positional scan approach was therefore able to select tetrapeptide sequences with antagonist potencies at the mMC4R similar to the endogenous antagonist (AGRP) and the synthetically-developed (SHU9119), indicating the utility of this approach in identifying novel ligands with potent functional activity.

Of the remaining twenty-four tetrapeptides, eleven possessing the Ac-Aromatic-Aromatic-Aromatic-Basic-NH₂ substitution pattern did not possess measurable antagonist activity at the highest concentrations assayed (Table 3). Of the five remaining peptides with this pattern, 4 (COR1-29-2, pA₂=6.7), 25 (COR1-80, pA₂=6.3), 28 (COR1-95, pA₂=6.6), 30 (COR1-90, pA₂=5.6), and 36 (COR2-3, pA₂=5.8), no strong activity trends were apparent with position 1 and 3 substitutions. Within these five, DLys at position 4 resulted in the most potent mMC4R antagonist activity, which can be observed by comparing the paired tetrapeptides at position 4 [FIG. 9, the cluster of DLys and Arg compounds without a corresponding DNal(2′) substitution]. By contrast, the eight compounds with the Ac-Aromatic-Basic-Aromatic-Aromatic-NH₂ substitution pattern possessed mMC4R antagonist activity. Six compounds with this scaffold had pA₂ values between 6.3 and 6.7. The remaining two paired tetrapeptides possessed the sequence Ac-Xxx-Arg-Trp-DNal(2′)-NH₂, where Xxx was DNal(2′) [17 (COR1-60, pA₂=7.0)] or DPhe(pI) [41 (COR1-85), pA₂=7.6]. Although a basic charge in position 2 generally resulted in more potent mMC4R antagonist than a basic charge in position 4, the potency was still decreased compared to tetrapeptides possessing two basic charges.

Although the tetrapeptide sequences presented herein have not previously been identified, the tripeptide sequence Ac-DNal(2′)-Arg-Nal(2′) (MCL0020) was reported to possess 11.63 and 1115 nM binding affinities at the human (h)MC4R and hMC3R, respectively, and was unable to displace radiolabeled NDP-MSH at up to 10 μM concentrations at the hMC1R (Chaki, S., et al., Eur. J. Pharmacol. 2003, 474 (1), 95-101). This tripeptide sequence is contained within the tetrapeptides 22 (COR1-29-8), 23 (COR2-33), and 24 (MDE10-28) from the present study, with an additional DLys, DNal(2′) or Arg added at the fourth position, respectively. The increased potency of 22 (COR1-29-8, pA₂=7.8) and 24 (MDE10-28, pA₂=8.8) compared to 23 (COR2-33, pA₂=6.7) indicates the importance of a basic residue in the fourth position for mMC4R antagonist potency and demonstrates that specific residues in this position can modulate antagonist potency over 100-fold range.

A subsequent structure-activity relationship study around the MCL0020 tripeptide, utilizing L and D isomers of Phe, Nal(1′), and Nal(2′) at the first position and L- and DNal(2′) at the third position indicated a series of compounds with nanomolar to micromolar affinities at the hMC4R (Nozawa, D., et al., Chem. Pharm. Bull. 2007, 55 (8), 1232-1239). The most potent compounds possessed DNal(2′) at the first position and either L- or DNal(2′) at the third position (IC₅₀ values of 15.4 and 36.5 nM, respectively; Nozawa, D., et al., Chem. Pharm. Bull. 2007, 55 (8), 1232-1239). Similar to the MCL0020 tripeptide, the Ac-DNal(2′)-Arg-DNal(2′) sequence was in three tetrapeptides from the present study [13 (COR1-29-5), 14 (COR2-57), and 15 (COR2-93) possessing DLys, DNal(2′), and Arg at the fourth position, respectively]. The basic residues in the fourth position increased antagonist potency approximately 100-fold at the mMC4R [pA₂ values of 8.2 and 8.3 for DLys and Arg, respectively, compared to 6.3 for DNal(2′)] when combined with this tripeptide sequence. These data indicate the importance of the fourth position within the tetrapeptide sequence, which may not be apparent from these published tripeptide sequences.

Melanocortin-3 Receptor Activity: Although the forty tetrapeptides utilized in this study were selected based on predicted MC4R antagonist pharmacology, structure-activity relationship (SAR) trends for the mMC3R were also observed. At the highest concentration assayed (100 μM), eleven of the forty tetrapeptides produced a partial agonist response, up to 50% of the maximal signal generated by NDP-MSH. Like at the mMC4R, no compound produced an agonist dose-response curve at the mMC3R.

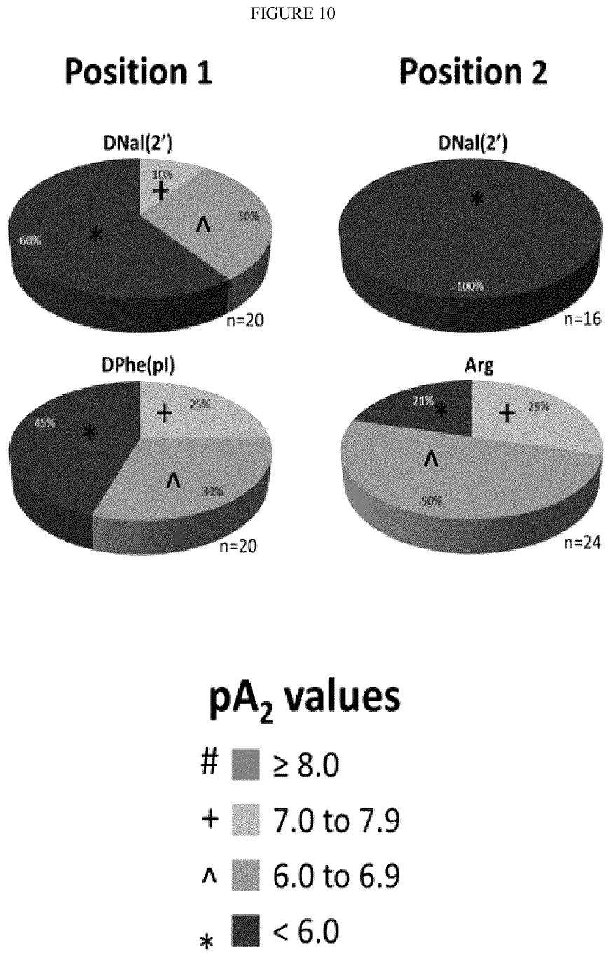

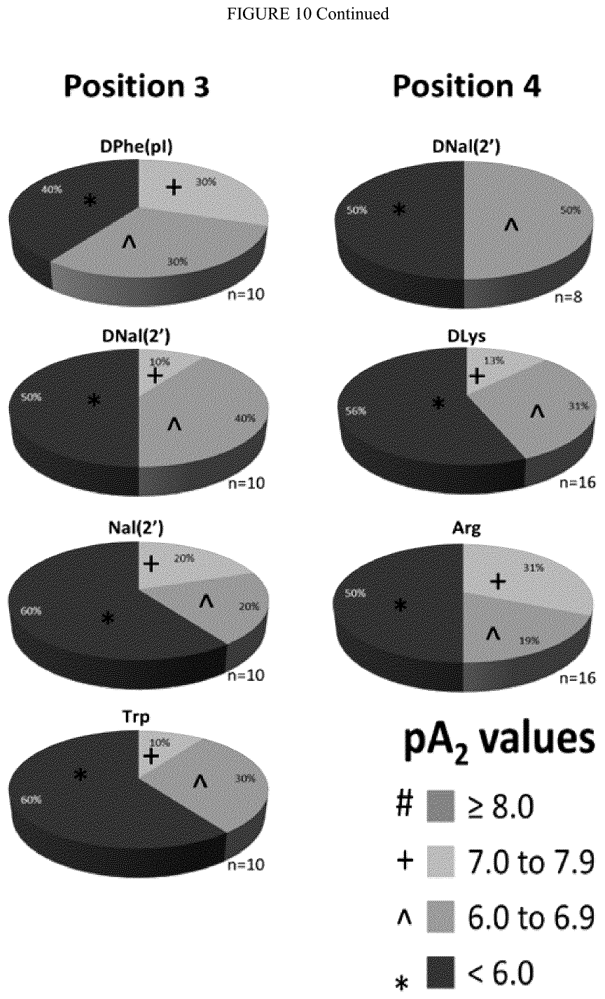

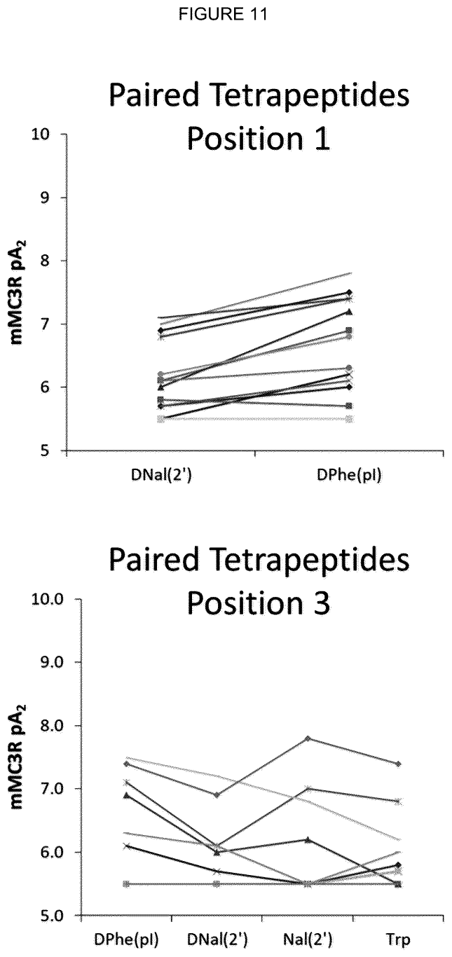

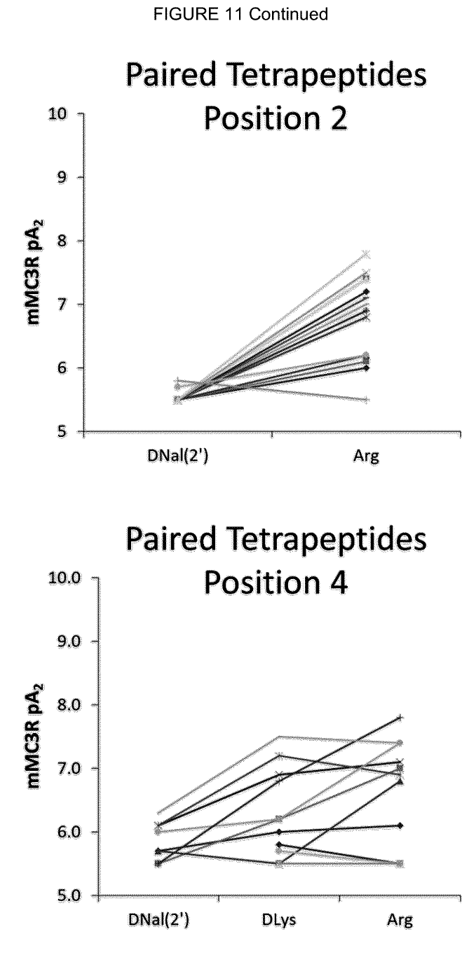

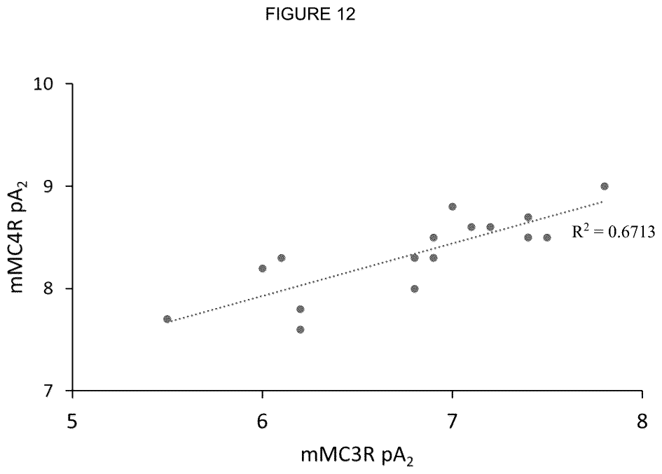

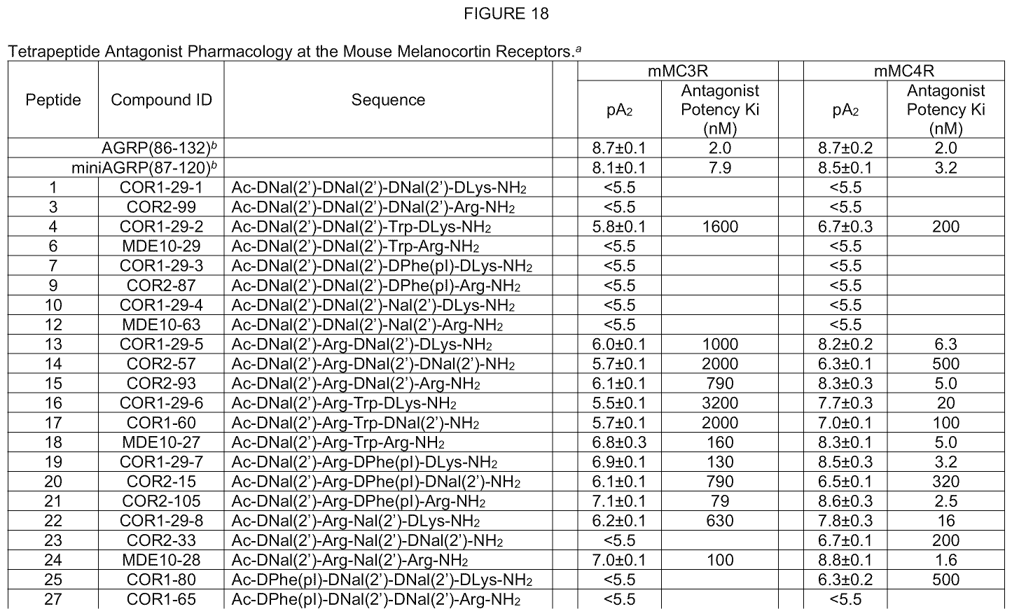

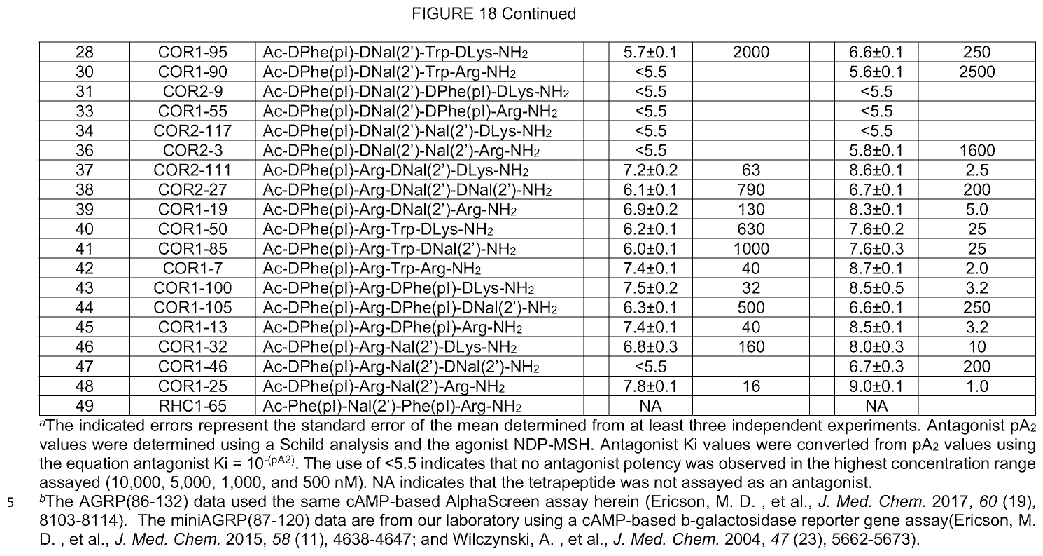

The sixteen tetrapeptides possessing the Ac-Aromatic-Basic-Aromatic-Basic-NH₂ substitution pattern all possessed measurable mMC3R antagonist activity, albeit with decreased potency when compared to the mMC4R (pA₂ ranges of 5.5 to 7.8 versus 7.6 to 9.0, respectively). The most potent MC3R antagonist, 48 (COR1-25, FIG. 4 and FIG. 18, pA₂=7.8), was the same tetrapeptide that resulted in the most potent MC4R antagonist. Similar to the mMC4R antagonist data, the importance of a basic residue in positions 2 and 4 can be seen in the pie charts comparing the different substitutions and mMC3R pA₂ values (FIG. 10). Compounds containing a DNal(2′) in position 2 all possessed pA₂ values less than 6, and tetrapeptides with DNal(2′) in position 4 all had pA₂ values less than 7. In contrast, seven compounds with Arg in position 2 and either a DLys or Arg in position 4 had pA₂ values between 7.0 and 7.8, demonstrating the most potent mMC3R antagonist tetrapeptides had two basic residues. Comparing paired tetrapeptides at positions 2 and 4 (FIG. 11), also indicates the relative importance of a basic charge compared to the aromatic DNal(2′). For compounds with the Ac-Aromatic-Basic-Aromatic-Basic-NH₂ pattern, there was a general correlation between mMC3R and mMC4R antagonist potencies (FIG. 12).

Of the sixteen compounds possessing the Ac-Aromatic-Aromatic-Aromatic-Basic-NH₂ substitution, fourteen did not produce measurable antagonist activity at the mMC3R at the concentrations assayed. The two remaining tetrapeptides from this pattern, 4 (COR1-29-2, pA₂=5.8) and 28 (COR1-95, pA₂=5.7), possessed measurable mMC4R antagonist activity and both contained DNal(2′) in position 2, Trp in position 3, and DLys in position 4. The eight remaining compounds possessed an Ac-Aromatic-Basic-Aromatic-Aromatic-NH₂ substitution pattern. Two were inactive at the concentrations assayed at the mMC3R [the paired tetrapeptides 23 (COR2-33) and 47 (COR1-46)]. The remaining six possessed pA₂ values between 5.7 to 6.3.

All tetrapeptides with measurable antagonist activity were more potent at the mMC4R compared to the mMC3R, indicating that no ligands were selective for mMC3R antagonism. While the majority of the ligands were less than 100-fold selective for the mMC4R over the mMC3R, three compounds were more than 150-fold selective for the mMC4R [13 (COR1-29-5), 15 (COR2-93), and 16 (COR1-29-6)]. Whereas AGRP-derived macrocyclic octapeptide antagonists have been described as 600- to 800-fold more selective for the mMC4R over the mMC3R (Fleming, K. A., et al., J. Med. Chem. 2018, 61 (17), 7729-7740), the relative facile synthesis of linear tetrapeptides versus head-to-tail cyclized macrocyclic octapeptides may afford a simpler scaffold to perform SAR campaigns, generating more potent and/or selective ligands and probe molecules. A potential method to identify more selective compounds would be to perform the initial mixture screen at both the MC3R and MC4R, and select ligands for individual compound synthesis based upon potential putative potency differences.

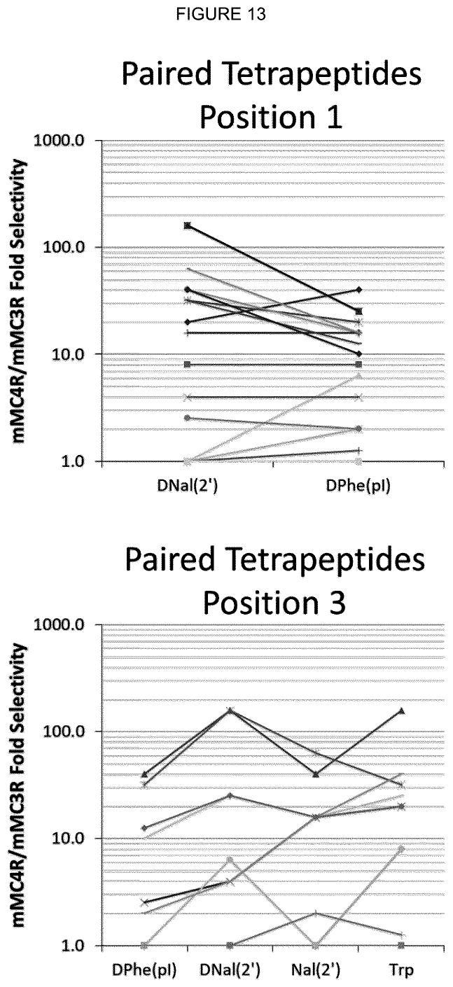

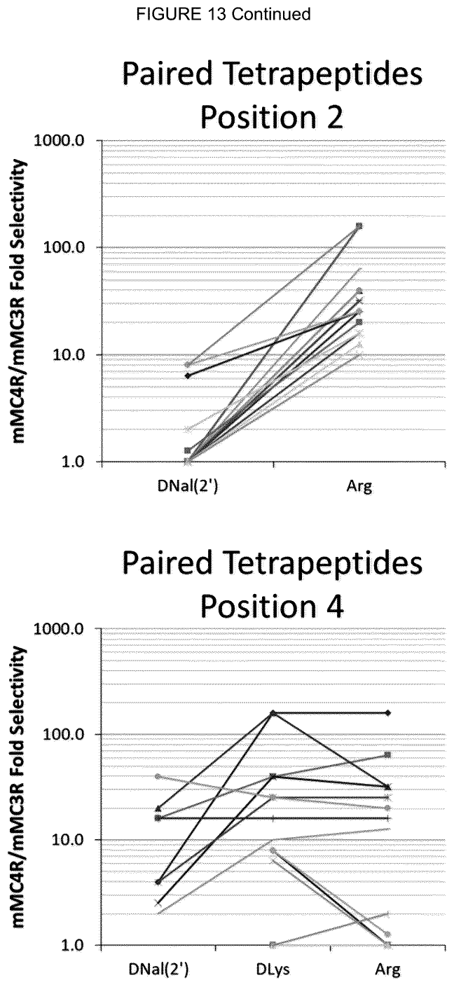

Certain residues appeared to drive the mMC4R over the mMC3R. After converting pA₂ to antagonist Ki values [antagonist Ki=10^{-(pA2)}], antagonist Ki values could be compared between paired compounds at the mMC4R and mMC3R (FIG. 13). Neither the DNal(2′) or DPhe(pI) substitution at position 1 resulted in a uniform trend. At position 2, every Arg substituted tetrapeptide possessed increased mMC4R selectivity compared to DNal(2′). While there is not an overall trend between all the substitutions at position 3, mMC4R selectivity was uniformly increased when DNal(2′) was incorporated versus DPhe(pI). Basic residues in position 4 also trended towards increased mMC4R selectivity versus DNal(2′)-substituted ligands. These data suggest certain residues can be incorporated to increase mMC4R antagonist selectivity over the mMC3R within the tetrapeptide scaffold.

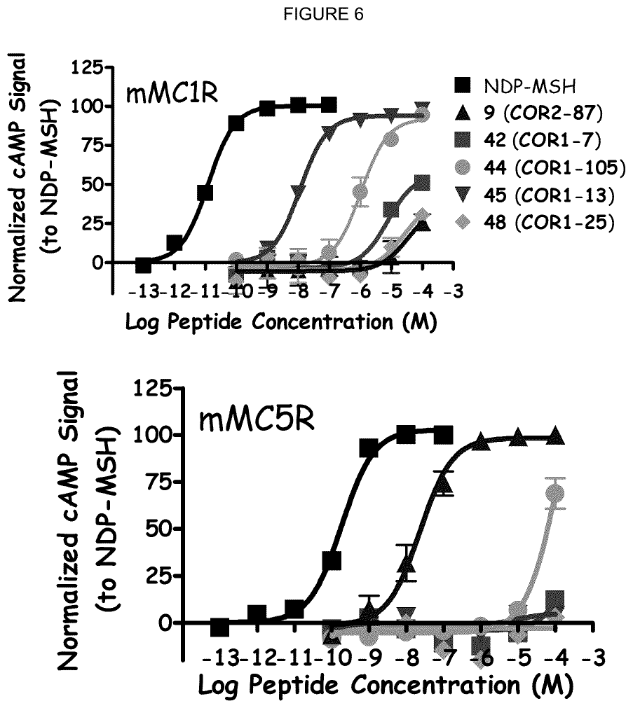

Melanocortin-1 Receptor Activity: Like the mMC3R data, SAR information can be obtained from the mMC1R data, although for this receptor a different substitution pattern was found to be important for activity. Of the forty tetrapeptides assayed at the mMC1R, six possessed full agonist activity [19 (COR1-29-7), 20 (COR2-15), 21 (COR2-105), 43 (COR1-100), 44 (COR1-105), and 45 (COR1-13)]. The full agonist efficacy of 45 (COR1-13) is presented in FIG. 6. Common to each of these tetrapeptides is an Ac-Xxx-Arg-DPhe(pI)-Yyy-NH₂ motif that inverts the natural Phe-Arg position of the endogenous agonist sequence His-Phe-Arg-Trp. The Ac-Xxx-Arg-DPhe(pI)-Yyy-NH₂ motif has previously been described as possessing full agonist activity at the mMC1R (Doering, S. R., et al., J. Med. Chem. 2017, 60 (10), 4342-4357; Fleming, K. A., et al., J. Med. Chem. 2019, 62 (5), 2738-27491; Schlasner, K. N., et al., Molecules 2019, 24 (8), 1463). In particular, the pharmacology of 20 (COR2-15) has previously been reported as an agonist at the mMC1R (EC₅₀=4,000 nM) with partial agonist efficacy at the mMC3R, mMC4R, and mMC5R (40%, 37%, and 43% of the maximal NDP-MSH signal, respectively), and as an antagonist at the mMC3R (pA₂=5.6) and mMC4R (pA₂=5.9) (Doering, S. R., et al., J. Med. Chem. 2017, 60 (10), 4342-4357). These values are similar to the agonist activity at the mMC1R (EC₅₀=5,000 nM), partial agonist efficacy at the mMC3R, mMC4R, and mMC5R (40%, 30%, and 40% of the maximal NDP-MSH signal, respectively), and antagonist activity at the mMC3R (pA₂=6.1) and mMC4R (pA₂=6.5) reported herein [Tables 2 and 3, 20 (COR2-15)]. Within this set of full mMC1R agonist tetrapeptides, a basic amino acid (DLys or Arg) at position 4 increased agonist potency more than 10-fold compared to an aromatic [DNal(2′)] residue.

All the full agonists at the mMC1R possessed a DPhe(pI) group in the third position. Select tetrapeptides containing other aromatic amino acids, including DNal(2′) [13 (COR1-29-5), 14 (COR2-57), 15 (COR2-93), 37 (COR2-111), and 39 (COR1-19)], Nal(2′) [22 (COR1-29-8)], or Trp [42 (COR1-7, FIG. 6)], in the third position resulted in partial efficacy agonists at the mMC1R (30-85% maximal NDP-MSH signal, EC₅₀ values 400-7,900 nM). Furthermore, while five of the six tetrapeptides with the Ac-Xxx-Arg-DNal(2′)-Yyy-NH₂ scaffold resulted in agonists with partial efficacy at the mMC1R, only one of the six tetrapeptides with either the Ac-Xxx-Arg-Nal(2′)-Yyy-NH₂ or the Ac-Xxx-Arg-Trp-Yyy-NH₂ sequences generated a partial mMC1R agonist response. The sigmoidal dose-response curve observed for eleven of the twelve tetrapeptides with a D-aromatic residue [DPhe(pI) or DNal(2′)] in the fourth position indicates that both the stereochemistry and functional group may be important for activation of the mMC1R.

Melanocortin-5 Receptor Activity: While the tetrapeptides were not selected for mMC5R activity, select SAR trends were observed at this receptor. The sixteen compounds with the Ac-Aromatic-Basic-Aromatic-Basic-NH₂ substitution pattern, important for mMC3R and mMC4R antagonist activity, did not possess mMC5R agonist potency at up to 100 μM concentrations. Of the eight compounds with the Ac-Aromatic-Basic-Aromatic-Aromatic-NH₂ motif, three were inactive. The other five [14 (COR2-57), 20 (COR2-15), 23 (COR2-33), 38 (COR2-27), and 44 (COR1-105, FIG. 6)] were able to partially stimulate the mMC5R at 100 μM concentrations (20% to 65% of the maximal NDP-MSH signal, FIG. 17). The sixteen remaining compounds had the Ac-Aromatic-Aromatic-Aromatic-Basic-NH₂ pattern. Only one of the eight compounds in this set with DLys at position 4 [34, (COR2-117)] resulted in partial activation of the mMC5R at 100 μM (50% of the maximal NDP-MSH signal). The other seven tetrapeptides were inactive at the highest assayed concentrations. Of the eight Ac-Aromatic-Aromatic-Aromatic-Basic-NH₂ tetrapeptides with Arg in position 4, one was inactive [30 (COR1-90)] and two [6 (MDE10-29) and 12 (MDE10-63)] partially stimulated the mMC5R at the highest concentrations assayed (45% and 40% of the maximal NDP-MSH signal, respectively). The remaining five compounds [3 (COR2-99), 9 (COR2-87), 27 (COR1-65), 33 (COR1-55), and 36 (COR2-3)] were full mMC5R agonists with sub-micromolar potencies (EC₅₀ between 30 and 800 nM). The nanomolar mMC5R potency of 9 (COR2-87) can be observed in FIG. 6. These compounds either partially activated or were inactive as agonists at the highest assayed concentrations at the mMC1R, mMC3R, and mMC4R, and did not act as antagonists at the mMC3R. Only one of these compounds, 36 (COR2-3), possessed mMC4R antagonist activity (pA₂=5.8). With the minimal observed activity at the other melanocortin receptors, the Ac-Aromatic-Aromatic-Aromatic-Arg-NH₂ motif may be further optimized for mMC5R agonist activity.

As an initial approach to identify the key residues for mMC5R selective agonism, an L-residue scan was performed on the 33 (COR1-55) tetrapeptide [Ac-DPhe(pI)-DNal(2′)-DPhe(pI)-Arg-NH_{2]} in which the D amino acids in the first three positions were systematically replaced with the L-isomers. While 33 (COR1-55) compound containing three D-aromatic residues was soluble and could be purified, the six tetrapeptides containing one or two D-amino acids were not soluble and could not be purified. The all-L tetrapeptide [49 (RHC₁-65), Ac-Phe(pI)-Nal(2′)-Phe(pI)-Arg-NH_{2]} was able to partially stimulate the mMC1R and was inactive at the mMC3R and mMC4R up to 100 μM concentrations, similar to 33 (COR1-55), but did not possess agonist activity at the mMC5R (FIG. 17). These data suggest that for this analog, the D-stereochemistry in the first three positions is preferred over the L-sterochemistry for the observed mMC5R-selective agonism, which may be utilized in the development of future tetrapeptide probes.

Potent MC5R agonists have been reported in the past. The mixed pharmacology of SHU9119 (Ac-Nle-c[Asp-His-DNal(2′)-Arg-Trp-Lys]-NH₂; MC1R/MC5R agonist, MC3R/MC4R antagonist) was first reported in 1995 (Hruby, V. J., et al., J. Med. Chem. 1995, 38 (18), 3454-3461). Replacing the His within the SHU9119 scaffold with Pro, resulting in the cyclic peptide Ac-Nle-c[Asp-Pro-DNal(2′)-Arg-Trp-Lys]-NH₂ (PG901), was reported to possess sub-nanomolar agonist potency at the hMC5R (Grieco, P., et al., J. Med. Chem. 2002, 45 (24), 5287-5294; and Grieco, P., et al., Biochem. Biophys. Res. Commun. 2002, 292 (4), 1075-1080). Similar to SHU9119, PG901 was reported to possess nanomolar potent antagonist activity at the hMC3R and hMC4R (pA₂=9.0 and 9.3, respectively) (Grieco, P., et al., J. Med. Chem. 2002, 45 (24), 5287-5294; Grieco, P., et al., Biochem. Biophys. Res. Commun. 2002, 292 (4), 1075-1080). While activity at the MC1R was not reported, due to the similar structure of PG901 to SHU9119 and similar pharmacology at the hMC3R, hMC4R, and hMC5R, PG901 may also have potent agonist activity at the MC1R. Potential advantages to the tetrapeptides reported herein may be synthetic tractability due to the lack of the lactam bridge, and greater selectivity for the MC5R over the other melanocortin receptors. Dosing of these compounds may be beneficial since they do not possess antagonist activities at the MC3R and MC4R, and may more clearly elucidate the in vivo functions of the MC5R, albeit with decreased potency compared to PG901.

Substitutions of the linear NDP-MSH have also been reported to result in selective and potent MC5R agonists. A sub-nanomolar MC5R agonist was identified (Ac-Ser-Tyr-Ser-Nle-Glu-Oic-DBip-Pip-Trp-Gly-Lys-Pro-Val-NH₂) that minimally activated the other melanocortin receptors at 5 μM, and possessed greater than 3200-fold selectivity for the MC5R as assessed by radiolabeled ¹²⁵I-NDP-MSH displacement (Bednarek, M. A., et al., Peptides 2007, 28 (5), 1020-1028). Truncation to Ac-Oic-DBip-Pip-Trp-NH₂ resulted in micromolar agonist potency at the MC5R (1.2 μM) (Bednarek, M. A., et al., Peptides 2007, 28 (5), 1020-1028). A similar substitution pattern of the His-DNal(2′)-Arg tripeptide sequence in SHU9119 with Oic-DBip-Pip, resulting in Ac-Nle-c[Asp-Oic-DBip-Pip-Trp-Lys]-NH₂, also resulted in potent and selective MC5R agonism (Bednarek, M. A., et al., J. Med. Chem. 2007, 50 (10), 2520-2526). This compound minimally activated the MC1R, MC3R, and MC4R (up to 40% at 2 μM) while activating the MC5R at a 0.99 nM concentration (Bednarek, M. A., et al., J. Med. Chem. 2007, 50 (10), 2520-2526). Additionally, this compound displaced radiolabeled NDP-MSH at the MC5R at a 0.95 nM concentration, but was unable to displace NDP-MSH at up to 5,000 nM concentrations at the MC1R, MC3R, and MC4R (Bednarek, M. A., et al., J. Med. Chem. 2007, 50 (10), 2520-2526). These results indicate that inserting an active MC5R tetrapeptide sequence into a linear or cyclically constrained scaffold can increase potency and/or selectivity. The most potent tetrapeptides from the present study at the MC5R [9 (COR2-87), 3 (COR2-99), and 33 (COR1-55); EC₅₀=30, 130, and 130 nM, respectively] were more potent than the equivalent Ac-Oic-DBip-Pip-Trp-NH₂ sequence (1.2 μM), suggesting that insertion of this new tetrapeptide into the NDP-MSH or MTII/SHU9119 scaffold may also increase MC5R agonist potency/selectivity.

### Conclusions

To identify novel MC4R antagonist sequences, an unbiased functional mixture-based positional scanning approach was utilized in an antagonist assay paradigm. Initially, mixtures comprising a library of 12,960,000 compounds were screened in the absence and presence of 0.5 nM NDP-MSH at a 50 μg/mL concentration. Twenty-six mixtures were then screened using two antagonist concentrations (25 and 50 μg/mL) with NDP-MSH (0.5 nM). Following library deconvolution, the defined amino acids of the most active mixtures at each position were selected for generating a library of forty-eight individual compounds. From this library, eight compounds (consisting of four aromatic amino acids, an acetylated N-terminal, and an amidated C-terminal) were not soluble in the purification solvents and were not advanced. The remaining forty compounds were screened for agonist activity at the mMC1R, mMC3R, mMC4R, and mMC5R. Compounds that did not stimulate the mMC3R and mMC4R were then screened as antagonists at these receptors with NDP-MSH serving as the agonist. Thirteen nanomolar potent mMC4R antagonist tetrapeptides were identified, with a general sequence of Ac-Aromatic-Basic-Aromatic-Basic-NH₂. One tetrapeptide (48, COR1-25) was identified that is about equipotent to the endogenous AGRP(86-132) antagonist (consisting of 46 amino acids and 5 disulfide bridges) at the MC4R, providing a potential new molecular probe for in vitro and in vivo studies. Three compounds possessed greater than 100-fold selectivity for the mMC4R over the mMC3R. A set of six full mMC1R agonists were identified with an inverted Arg-Phe motif. Five sub-micromolar mMC5R agonist tetrapeptides were also reported with minimal activation of the remaining receptors. Compounds from this library may prove useful in generating novel probes and therapeutic leads for the treatment of disease states of negative energy balance, including cachexia associated with cancer.

### Experimental

Mixture-Based Positional Scanning Library: As previously described (Haslach, E. M., et al., J. Med. Chem. 2014, 57 (11), 4615-4628; Doering, S. R., et al., J. Med. Chem. 2017, 60 (10), 4342-4357; Houghten, R. A., Proc. Natl. Acad. Sci. U S. A. 1985, 82 (15), 5131-5135; Ostresh, J. M., et al., Biopolymers 1994, 34 (12), 1681-1689; and Houghten, R. A., et al., Int. J. Pept. Protein Res. 1986, 27 (6), 673-678), the TPI924 library was synthesized using the simultaneous multiple-peptide synthesis approach. This TPI924 Ac-tetrapeptide-NH₂ template [Ac-A¹—A²—A³—A⁴—NH₂] library contains 12,960,000 tetrapeptides in 240 mixtures composed of 60 amino acid building block combinations at each of the “R” positions. The sixty amino acids used for each of the “A” positions within the tetrapeptide template include Ala, Asp, Glu, Phe, Gly, His, Ile, Lys, Leu, Met, Asn, Pro, Gln, Arg, Ser, Thr, Val, Trp, Tyr, DAla, DAsp, DGlu, DPhe, DHis, DIle, DLys, DLeu, DMet, DAsn, DPro, DGln, DArg, DSer, DThr, DVal, DTrp, DTyr, Nle, DNle, Cha, DCha, Ala(3-Pyr), DAla(3-Pyr), Ala(2-Thi), DAla(2-Thi), Tic, DTic, Phe(pCl), DPhe(pCl), Phe(pI), DPhe(pI), Phe(pNO2), DPhe(pNO2), Nal(2′), DNal(2′), E-aminocaproic acid, Met[O2], dehydPro, and Tyr(3-I).

Fluorescent β-Galactosidase Assay: A fluorescent β-galactosidase bioassay was used for the primary screen of the mixture-based positional scanning library TPI924, based upon an absorbance β-galactosidase bioassay previously described (Chen, W. B., et al., Anal. Biochem. 1995, 226 (2′), 349-354). HEK-293 cells stably expressing the mMC4R were transfected with 4 μg of CRE/β-galactosidase using the calcium phosphate method (Chen, C. A., et al., BioTechniques 1988, 6 (7), 632-638). After 24 h, cells were plated onto collagen-treated black 96-well plates (Corning) and incubated at 37° C. with 5% CO₂. At 48 h post-transfection, the cell media was aspirated and the cells were stimulated with compounds mixtures, in both agonist and antagonist experimental paradigms. Compound mixtures were dissolved 1:1 DMF:H₂O at 20 mg/mL stock concentrations, diluted with H₂O to 2 mg/mL working concentrations, and stored at −20° C. until use. For fluorescence agonist experiments, 40 μL of the TPI924 peptide mixture (50 μg/mL) in assay media (DMEM containing 0.1 mg/mL BSA and 0.1 mM isobutylmethylxanthine) was added to each well. For fluorescence antagonist studies, 40 μL of the TPI mixture (50 μg/mL) and NDP-MSH (0.5 nM) in assay media was added to each well. Triplicate wells were used for each experimental mixture, and at least three independent experiments were performed. Controls included NDP-MSH (10⁻⁶ to 10⁻¹³ M), forskolin (100 nM), and assay media. The plates were incubated at 37° C. with 5% CO₂ for 6 h. Post-stimulation, the media was aspirated and 120 μL of lysis buffer (7:1 Z-buffer [60 mM Na₂PO₄.7H₂O, 40 mM Na₂H₂PO₄.H₂O, 10 mM KCl, 1 mM MgSO₄.7H₂O, pH=7.0]:1% Triton X-100 in H₂O [v/v]) was added. Plates were stored at −80° C. for up to one week.

The plates were thawed and 10 μL aliquots of cell lysate were added to 200 μL of BioRad dye solution (1:4 dye solution:H₂O) in another clear 96-well plate. Absorbance was read using a FlexStation 3 (Molecular Devices) at λ=595 nm. To the remaining cell lysate, 30 μL of substrate buffer [0.1 mM 4-methylumbelliferyl-β-D-galactopyranoside (4-MUG), 0.045 M β-mercaptoethanol, dissolved in Z-buffer] was added to each well, and the plates incubated at 37° C. One plate was monitored in fluorescence mode (excitation λ=350 nm, emission λ=450 nm), until the positive controls reached 8,000 RFU, at which point 75 μL of the stop buffer (300 mM glycine, 15 mM EDTA, pH 11.2) was added to each well. Fluorescence readings were measured on a FlexStation 3 (Molecular Devices) in Endpoint Fluorescence mode (excitation λ=350 nm, emission λ=450 nm). For each well, the plate was normalized to the corresponding well protein level, plate basal signal, and the maximal NDP-MSH signal (10⁻⁶ to 10⁻⁸M). For agonists screens, the normalized well signal was converted to a percentage from plate basal signal to the plate maximal NDP-MSH response. In the initial antagonist screen, the normalized well signal was converted to a percentage from the median plate experimental signal to basal activity. In the two-point follow-up antagonist screen, the normalized well signal was converted to a percentage from plate 0.5 nM NDP-MSH signal to basal activity. Each mixture was screened using triplicate wells in at least two independent experiments. Agonist and antagonist means were calculated from each mixture replicate assays. Singleton outlying points were eliminated using the quartile method (NIST/SEMATECH e-Handbook of Statistical Methods. http://www.itl.nist.gov/div898/handbook/ (accessed Jun. 29, 2021)).

Tetrapeptide Synthesis and Purification: The amino acids Fmoc-Arg(Pbf), Fmoc-Trp(Boc), and Fmoc-DLys(Boc), the Rink-amide MBHA resin, and coupling reagent 2-(1H-benzotriazol-1-yl)-1,1,3,3-tetramethyluronium hexafluorophosphate (HBTU) were purchased from Peptides International (Louisville, Ky.). The Fmoc-DNal(2′) amino acid was purchased from Bachem and Peptides International. The Fmoc-Nal(2′) residue was purchased from SyntheTech. The Fmoc-DPhe(pI) amino acid was purchased from Alfa Aesar. Dichloromethane (DCM), methanol (MeOH), acetonitrile (ACN), dimethylformamide (DMF), and anhydrous diethyl ether were purchased from Fisher (Fair Lawn, N.J.). Trifluoroacetic acid (TFA), dimethyl sulfoxide (DMSO), piperidine, triisopropylsilane (TIS), thioanisole, and N,N-diisopropylethylamine (DIEA) were purchased from Sigma-Aldrich (St. Louis, Mo.). All reagents and chemicals were ACS grade or better and were used without further purification.

The tetrapeptides identified from the library deconvolution were synthesized using standard N-α-fluorenylmethoxycarbonyl (Fmoc) methodologies (Carpino, L. A., et al., J. Am. Chem. Soc. 1970, 92 (19), 5748-5749; and Carpino, L. A., et al., J. Org. Chem. 1972, 37 (22), 3404-3409), individually on a microwave synthesizer (Discover SPS; CEM, Matthews, N.C.) or in parallel (LabTech I; Advanced ChemTech, Louisville, Ky.). The resin was allowed to swell in DCM before iterative Fmoc deprotection and amino acid coupling steps were used to assemble the desired tetrapeptides. For peptides synthesized on the microwave synthesizer, the Fmoc group was removed using a two-step deprotection strategy with 20% piperidine in DMF (1×2 min at room temperature, followed by 1×4 min using microwave irradiation to 75° C., and 30 W). Microwave coupling reactions were carried out at 75° C. and 30 W for 5 min (10 min for coupling Arg residues). For peptides synthesized in a parallel, a two-step deprotection strategy was used (1×5 min, 1×15 min at room temperature) with 20% piperidine in DMF. Parallel coupling reactions were carried out at room temperature for 45 min. For most coupling reactions, 3.1 eq of the amino acid, 3 eq HBTU, and 5 eq of DIEA were used. For Arg coupling, high equivalents were employed (5.1 eq Arg, 5 eq HBTU, and 7.1 eq DIEA). In all cases, reaction progress was monitored using a ninhydrin assay (Kaiser, E., et al., Anal. Biochem. 1970, 34 (2), 595-598), and deprotection and coupling reactions were repeated if necessary. Following the removal of the terminal Fmoc group, tetrapeptides were acetylated by adding a 3:1 acetic anhydride:pyridine solution and mixing for 30 min at room temperature. The resin was then washed with DMF, DCM, and MeOH. Tetrapeptides were cleaved from resin and side-chain deprotected using a 91:3:3:3 mixture of TFA:H₂O: TIS:thioanisole for 2 h at room temperature, and then precipitated using ice-cold diethyl ether. Crude peptides were pelleted on a Sorvall Legend XTR centrifuge, and dried overnight in a vacuum desiccator.

The crude tetrapeptides were purified on a C18 RP-HPLC semipreparative column (Vydac 218TP1010, 1.0 cm x 25 cm) using a Shimadzu system equipped with a UV detector. Peptides are ≥95% pure as ascertained by analytical RP-HPLC (Vydac 218TP104, 0.46 cm×25 cm) on a Shimadzu system equipped with a PDA detector in two diverse solvent systems (methanol and acetonitrile). The peptides possessed the correct average molecule mass by ESI-MS (Bruker BioTOF II ESI/TOF-MS; LeClaire-Dow Instrumentation Facility, University of Minnesota). cAMP AlphaScreen Bioassay: The purified tetrapeptides were dissolved in DMSO at a stock concentration of 10⁻²M (NDP-MSH in H₂O at a stock concentration of 10⁴ M), and assayed using HEK293 cells stably expressing the mouse MC1R, MC3R, MC4R, and MC5R using the AlphaScreen cAMP bioassay (PerkinElmer) according to the manufacturer's instructions and as previously described (Singh, A., et al., ACS Med. Chem. Lett. 2015, 6 (5), 568-572; Ericson, M. D., et al., Bioorg. Med. Chem. Lett. 2015, 25 (22), 5306-5308; and Lensing, C. J., et al., J. Med. Chem. 2016, 59 (7), 3112-3128).

Briefly, cells 70-90% confluent were dislodged with Versene (Gibco) at 37° C. and plated 10,000 cells/well in a 384-well plate (Optiplate) with 10 μL of freshly prepared stimulation buffer (1× HBSS, 5 mM HEPES, 0.5 mM IBMX, 0.1% BSA, pH=7.4) with 0.5 μg of anti-cAMP acceptor beads per well. The cells were stimulated with the addition of 5 μL of stimulation buffer containing peptide (concentrations from 10-4 to 10-13 M, determined by ligand potency) or forskolin (10-4 M) and incubated in the dark at room temperature for 2 hours.

Following stimulation, streptavidin donor beads (0.5 μg) and biotinylated-cAMP (0.62 μmol) were added to the wells in a green light environment with 10 μL of lysis buffer (5 mM HEPES, 0.3% Tween-20, 0.1% BSA, pH=7.4) and the plates were incubated in the dark at room temperature for an additional 2 h. Plates were read on a Enspire (PerkinElmer) Alpha-plate reader using a pre-normalized assay protocol (set by the manufacturer).

Data Analysis: The pA₂ and EC₅₀ values represent the mean of at least three independent experiments performed in duplicate replicates. Compounds that were not active in two independent experiments at the concentrations assayed (>100 μM for agonist assays; 10,000, 5,000, 1,000, and 500 nM for antagonist assays) were not furthered examined. The pA₂ and EC₅₀ estimates and associated standard errors (SEM) were determined by fitting the data to a nonlinear least-squares analysis using the PRISM program (version 4.0, GraphPad Inc). The peptides were assayed as TFA salts and not corrected for peptide content.

### Example 2

### Characterization of LTT1-20 (COR1-25) Agonists and Antagonists

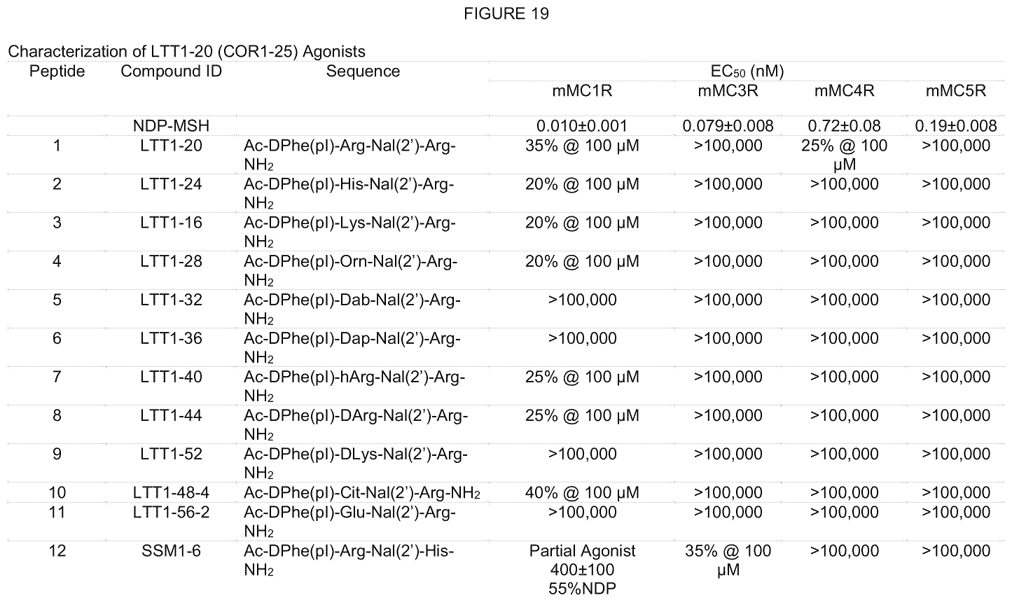

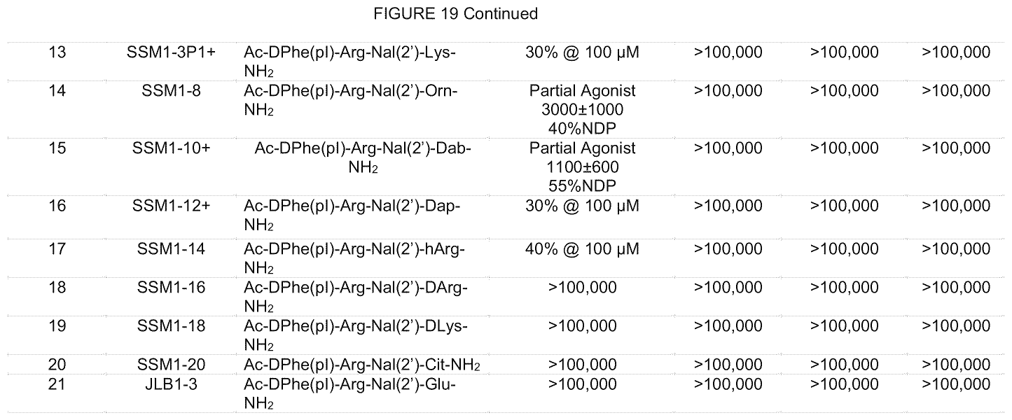

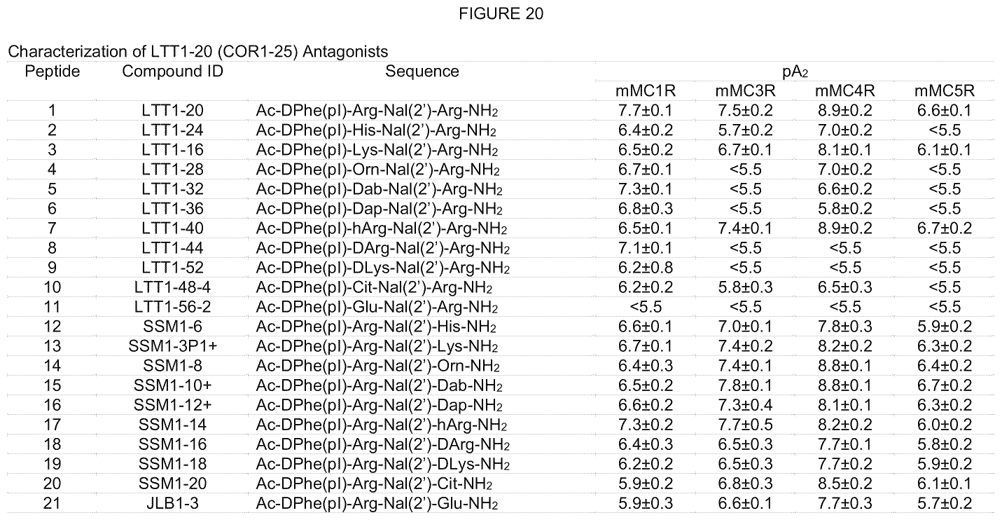

FIGS. 19 and 20 characterize analogs of COR1-25 (LTT1-20), there the A² or A⁴ Arg is substituted with His, Lys, Orn, Dab, Dap, hArg, DArg, DLys, Cit, or Glu. These analogs were prepared and tested according to the same methods described in Example 1.

All publications, patents, and patent documents are incorporated by reference herein, as though individually incorporated by reference. The invention has been described with reference to various specific and preferred embodiments and techniques. However, it should be understood that many variations and modifications may be made while remaining within the spirit and scope of the invention.

What is claimed is:

1. A compound of formula (I):
R¹—A¹—A²—A³—A⁴—N(R²)₂ (I)
wherein:
R¹ is H, (C₂-C₆)alkanoyl, (C₂-C₆)cycloalkyl or (C₂-C₄)alkyl, optionally substituted with cycloalkyl;
each R² is independently H or (C₂-C₆)alkyl;
A¹ is a residue of an aromatic D-amino acid;
A² is a residue of a basic amino acid;
A³ is a residue of an aromatic L- or a D-amino acid; and
A⁴ is a residue of an L- or D-amino acid.

2. The compound of claim 1, wherein A¹ is a residue of D-Nal(2′), D-Phe(pI), or D-Trp.

3. The compound of claim 1, wherein A² is a residue of Arg, His, Lys, Orn, Dab, Dap, hArg, D-Arg, D-Lys, or Cit.

4. The compound of claim 1 wherein A³ is a residue of D-Nal(2′), D-Phe(pI), D-Trp, L-Nal(2′), L-Phe(pI), or L-Trp.

5. The compound of claim 1, wherein A⁴ is a residue of a basic amino acid.

6. The compound of claim 5, wherein A⁴ is a residue of Arg, His, Lys, Orn, Dab, Dap, hArg, D-Arg, D-Lys, or Cit.

7. The compound of claim 1 selected from the group consisting of:
Ac-DNal(2′)-Arg-DNal(2′)-DLys-NH₂
Ac-DNal(2′)-Arg-DNal(2′)-Arg-NH₂
Ac-DNal(2′)-Arg-Trp-DLys-NH₂
Ac-DNal(2′)-Arg-Trp-Arg-NH₂
Ac-DNal(2′)-Arg-DPhe(pI)-DLys-NH₂
Ac-DNal(2′)-Arg-DPhe(pI)-Arg-NH₂
Ac-DNal(2′)-Arg-Nal(2′)-DLys-NH₂
Ac-DNal(2′)-Arg-Nal(2′)-Arg-NH₂
Ac-DPhe(pI)-Arg-DNal(2′)-DLys-NH₂
Ac-DPhe(pI)-Arg-DNal(2′)-Arg-NH₂
Ac-DPhe(pI)-Arg-Trp-DLys-NH₂
Ac-DPhe(pI)-Arg-Trp-Arg-NH₂
Ac-DPhe(pI)-Arg-DPhe(pI)-DLys-NH₂
Ac-DPhe(pI)-Arg-DPhe(pI)-Arg-NH₂
Ac-DPhe(pI)-Arg-Nal(2′)-DLys-NH₂
Ac-DPhe(pI)-Arg-Nal(2′)-Arg-NH₂

8. The compound of claim 1 selected from the group consisting of:
Ac-DPhe(pI)-His-Nal(2′)-Arg-NH₂
Ac-DPhe(pI)-Lys-Nal(2′)-Arg-NH₂
Ac-DPhe(pI)-Orn-Nal(2′)-Arg-NH₂
Ac-DPhe(pI)-Dab-Nal(2′)-Arg-NH₂
Ac-DPhe(pI)-Dap-Nal(2′)-Arg-NH₂
Ac-DPhe(PI)-hArg-Nal(2′)-Arg-NH₂
Ac-DPhe(pI)-DArg-Nal(2′)-Arg-NH₂
Ac-DPhe(pI)-DLys-Nal(2′)-Arg-NH₂
Ac-DPhe(pI)-Cit-Nal(2′)-Arg-NH₂
Ac-DPhe(pI)-Arg-Nal(2′)-His-NH₂
Ac-DPhe(pI)-Arg-Nal(2′)-Lys-NH₂
Ac-DPhe(pI)-Arg-Nal(2′)-Orn-NH₂
Ac-DPhe(pI)-Arg-Nal(2′)-Dab-NH₂
Ac-DPhe(pI)-Arg-Nal(2′)-Dap-NH₂
Ac-DPhe(pI)-Arg-Nal(2′)-hArg-NH₂
Ac-DPhe(pI)-Arg-Nal(2′)-DArg-NH₂
Ac-DPhe(pI)-Arg-Nal(2′)-DLys-NH₂
Ac-DPhe(pI)-Arg-Nal(2′)-Cit-NH₂

9. A pharmaceutical composition comprising a compound of formula (I) as described in claim 1 or a pharmaceutically acceptable salt thereof, and a pharmaceutically acceptable carrier.

10. A dietary supplement comprising a compound of formula (I) as described in claim 1 or a pharmaceutically acceptable salt thereof.

11. A method for increasing appetite, treating a disease of negative energy balance, or promoting analgesia in a mammal, comprising administering an effective amount of a compound of formula (I) as described in claim 1 or a pharmaceutically acceptable salt thereof, to the mammal.

12. A compound of formula (II):
R³—A⁵—A⁶—A⁷—A⁸—N(R⁴)₂ (II)
wherein:
R³ is H, (C₁-C₆)alkanoyl, (C₁-C₆)cycloalkyl or (C₁-C₄)alkyl, optionally substituted with cycloalkyl;
each R⁴ is independently H or (C₁-C₆)alkyl;
A⁵ is a residue of an aromatic D-amino acid;
A⁶ is a residue of an aromatic D-amino acid;
A⁷ is a residue of an aromatic L- or a D-amino acid; and
A⁸ is a residue of a basic amino acid.

13. The compound of claim 12, wherein A⁵ is a residue of D-Nal(2′), D-Phe(pI), or D-Trp.

14. The compound of claim 12, wherein A⁶ is a residue of D-Nal(2′), D-Phe(pI), or D-Trp.

15. The compound of claim 12, wherein A⁷ is a residue of D-Nal(2′), D-Phe(pI), D-Trp, L-Nal(2′), L-Phe(pI), or L-Trp.

16. The compound of claim 12, wherein A⁸ is a residue of Arg or DLys.

17. The compound of claim 12 selected from the group consisting of:
Ac-DNal(2′)-DNal(2′)-DNal(2′)-DLys-NH₂
Ac-DNal(2′)-DNal(2′)-DNal(2′)-Arg-NH₂
Ac-DNal(2′)-DNal(2′)-Trp-DLys-NH₂
Ac-DNal(2′)-DNal(2′)-Trp-Arg-NH₂
Ac-DNal(2′)-DNal(2′)-DPhe(pD-DLys-NH₂
Ac-DNal(2′)-DNal(2′)-DPhe(pD-Arg-NH₂
Ac-DNal(2′)-DNal(2′)-Nal(2′)-DLys-NH₂
Ac-DNal(2′)-DNal(2′)-Nal(2′)-Arg-NH₂
Ac-DPhe(pI)-DNal(2′)-DNal(2′)-DLys-NH₂
Ac-DPhe(pI)-DNal(2′)-DNal(2′)-Arg-NH₂
Ac-DPhe(pI)-DNal(2′)-Trp-DLys-NH₂
Ac-DPhe(pI)-DNal(2′)-Trp-Arg-NH₂
Ac-DPhe(pI)-DNal(2′)-DPhe(pI)-DLys-NH₂
Ac-DPhe(pI)-DNal(2′)-DPhe(pI)-Arg-NH₂
Ac-DPhe(pD-DNal(2′)-Nal(2′)-DLys-NH₂
Ac-DPhe(pI)-DNal(2′)-Nal(2′)-Arg-NH₂

18. A pharmaceutical composition comprising a compound of formula (II) as described in claim 12 or a pharmaceutically acceptable salt thereof, and a pharmaceutically acceptable carrier.

19. A dietary supplement comprising a compound of formula (II) as described in claim 12 or a pharmaceutically acceptable salt thereof.

20. A method for modulating muscle glucose uptake, secretion of oils in the skin, treating acne, or treating anxiety or depression in a mammal, comprising administering an effective amount of a compound of formula (II) as described in claim 12 or a pharmaceutically acceptable salt thereof, to the mammal.
